(19) United States
(12) Patent Application Publication
(10) Pub. No.: US 2010/0298415 A1
(43) Pub. Date: Nov. 25, 2010

(54) TARGETED SCHIFF BASE COMPLEXES

(75) Inventors: Allison S. Harney, Chicago, IL (US); Jiyoun Lee, Palo Alto, CA (US); Carole B. LaBonne, Evanston, IL (US); Thomas J. Meade, Wilmette, IL (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/729,814

(22) Filed: Mar. 23, 2010

Related U.S. Application Data
(60) Provisional application No. 61/162,481, filed on Mar. 23, 2009.

(51) Int. Cl. A61K 31/7088; C07H 21/04; C07K 14/00; A61P 35/00

(52) U.S. Cl. 514/44 R; 536/22.1; 536/23.1; 530/395

(57) ABSTRACT

The present invention provides targeted Schiff base complexes. In particular the present invention provides biopolymer targeted transition metal complexes configured to inhibit the activity of targeted proteins, methods of synthesis thereof, and pharmaceutical compositions and uses thereof.

The present application claims priority to U.S. Provisional Patent Application Ser. No. 61/162,481 filed Mar. 23, 2009, the entire disclosure of which is herein incorporated by reference in its entirety.

### FIELD OF THE INVENTION

The present invention provides targeted Schiff base complexes. In particular, the present invention provides biopolymer targeted transition metal complexes configured to inhibit the activity of targeted proteins, methods of synthesis thereof, and pharmaceutical compositions and uses thereof.

### BACKGROUND OF THE INVENTION

The normal growth, development, and function of an organism require precise and coordinated control of gene expression at the level of transcription. A major part of this control is exerted by interactions between an array of transcription regulatory proteins and specific regulatory DNA sequences. The combination of such proteins and DNA sequences is specific for given gene or group of genes in a particular cell type and the proteins regulating the same gene may vary between cell types. The expression or activity of these regulatory proteins may be modified during various states of cell differentiation, in response to an external stimulus, or in certain disease states. For example, cancer cells exhibit aberrant regulation of transcription for a variety of different genes. Re-regulation of transcription through the inhibition of specific transcription factors provides a mechanism for treatment or prevention of disease.

For example, breast cancer is a heterogeneous disease with fatalities resulting from recurrence and metastasis to distant sites including bone, lung, liver and brain (Kuperwasser C, et al. (2005) Cancer Res 65(14):6130-6138, herein incorporated by reference in its entirety). The zinc finger transcription factors Slug, Snail and Sip1 have been implicated in tumor metastasis through the regulation of epithelial-to-mesenchymal transitions (EMTs) (Savagner et al. (1997) J Cell Biol 137(6):1403-1419, Vandewalle et al. (2005) Nucleic Acids Res 33(20):6566-6578, Kuphal et al. (2005) Melanoma Res 15(4):305-313, Blanco et al. (2002) Oncogene 21(20):3241-3246, herein incorporated by reference in their entireties). Much of what is known about the molecular mechanism via which these transcriptional regulators mediate EMT is from developmental studies, particularly the induction of vertebrate neural crest migration (Sakai et al. (2006) Development 133(7):1323-1333, Cheung et al. (2005) Dev Cell 8(2):179-192, Mayor et al. (1995) Development 121(3):767-777, Aybar et al. (2003) Development 130(3):483-494, LaBonne & Bronner-Fraser (2000) Dev Biol 221(1):195-205, Carl et al. (1999) Dev Biol 213(1):101-115, herein incorporated by reference in their entireties). Xenopus laevis embryos have been used as a model system to study the molecular activities of Snail family members (Aybar et al. (2003) Development 130(3):483-494, LaBonne & Bronner-Fraser (2000) Dev Biol 221(1):195-205, Carl et al. (1999) Dev Biol 213(1):101-115, Vernon & Labonne (2006) Development 133(17):3359-3370, herein incorporated by reference in their entireties). During EMT, transcriptional repressors down-regulate the expression of proteins involved in cell-cell adhesions characteristic of epithelial cells. Proteins involved in invasion, such as matrix metalloproteinases, are upregulated and cells lose their epithelial characteristics becoming invasive mesenchymal cells (Shiozaki et al. (1996) Cancer 77(8 Suppl):1605-1613, Martinez-Estrada et al. (2006) Biochem J 394(Pt 2):449-457, Peinado (2004) Mol Cell Biol 24(1):306-319, Yang et al. (2005) Cancer Res 65(8):3179-3184, Peinado et al. (2004) Int J Dev Biol 48(5-6):365-375, herein incorporated by reference in their entireties). What is needed are new compositions and methods for treating and preventing cancer metastasis and recurrence by altering the cellular and molecular biology associated with such pathways and processes.

### SUMMARY OF THE INVENTION

The present invention provides targeted Schiff base complexes. In particular, the present invention provides biopolymer targeted transition metal complexes configured to inhibit the activity of targeted proteins, methods of synthesis thereof, and pharmaceutical compositions and uses thereof.

In some embodiments, the present invention provides a composition comprising: (a) a transition metal coordination element, (b) a linker element, and (c) a biopolymer element, wherein the coordinated transition metal is conjugated to the biopolymer by the linker. In some embodiments, a transition metal is coordinated by the transition metal coordination element.

In some embodiments, the present invention provides a composition comprising: (a) a biopolymer element; (b) a linker element, wherein the linker comprises a structure comprising Formula 1:

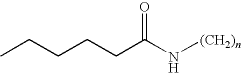

wherein n is an integer between 1 and 10, and wherein the ultimate CH₂ group provides an attachment site for the biopolymer element; and (c) a transition metal coordination element, wherein the transition metal coordination element comprises a structure comprising Formula 2:

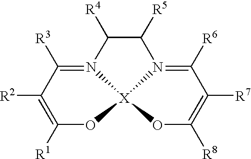

wherein: X comprises a transition metal; R₁ comprises hydrogen, alkyl, hydrophilic organic acid, alkyl amine, amine, alkyl alcohol, alcohol, aryl, or an attachment site for the linker element; R₂ comprises hydrogen, alkyl, hydrophilic organic acid, alkyl amine, amine, alkyl alcohol, alcohol, aryl, or an attachment site for the linker element; R₃ comprises hydrogen, alkyl, hydrophilic organic acid, alkyl amine, amine, alkyl alcohol, alcohol, aryl, or an attachment site for the linker element; R₄ comprises hydrogen, alkyl, hydrophilic organic acid, alkyl amine, amine, alkyl alcohol, alcohol, aryl, or an attachment site for the linker element; R₅ comprises hydrogen, alkyl, hydrophilic organic acid, alkyl amine, amine, alkyl alcohol, alcohol, aryl, or an attachment site for the linker element; R₆ comprises hydrogen, alkyl, hydrophilic organic acid, alkyl amine, amine, alkyl alcohol, alcohol, aryl, or an attachment site for the linker element; R₇ comprises hydrogen, alkyl, hydrophilic organic acid, alkyl amine, amine, alkyl alcohol, alcohol, aryl, or an attachment site for the linker element; and R₈ comprises hydrogen, alkyl, hydrophilic organic acid, alkyl amine, amine, alkyl alcohol, alcohol, aryl, or an attachment site for the linker element; and wherein at least one of R₁, R₂, R₃, R₄, R₅, R₆, R₇, and R₈, comprises an attachment site for the linker element; wherein the coordinated transition metal element is conjugated to the biopolymer by the linker. In some embodiments, the biopolymer comprises nucleic acid. In some embodiments, the nucleic acid comprises the binding site for a DNA binding protein. In some embodiments, R₅ comprises an attachment site for the linker, and each of R₁, R₂, R₃, R₄, R₆, R₇, and R₈ comprise hydrogen. In some embodiments, the coordinated transition metal is selected from nickel, cadmium, zinc, iron and cobalt. In some embodiments, the coordinated transition metal comprises cobalt. In some embodiments, the transition metal coordination element comprises a structure comprising Formula 3:

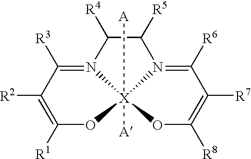

wherein A and A′ are axial ligands. In some embodiments, A and A′ comprise the same type of molecule or functional group. In some embodiments, A and A′ comprise different types of molecules or functional groups. In some embodiments, A and A′ are any one of 2-methyl imidazole, 4-methyl imidazole, imidazole, ammine, phosphate, analine, pyridine, cysteine, and courmarin. In some embodiments, the transition metal coordination element comprises a structure comprising Formula 4:

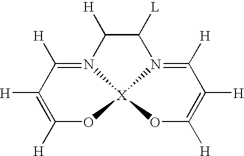

wherein L comprises the attachment site for the linker element. In some embodiments, the transition metal coordination element comprises a structure comprising Formula 5:

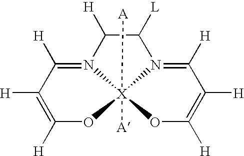

In some embodiments, the present invention provides a composition comprising a structure of Formula 6:

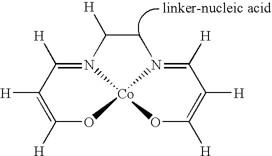

In some embodiments, the linker comprises the structure of Formula 1. In some embodiments, the nucleic acid comprises the binding site for a DNA binding protein. In some embodiments, the nucleic acid comprises the binding site for a transcription factor. In some embodiments, the nucleic acid comprises the binding site for Snail family transcription factors. In some embodiments, the nucleic acid comprises a first DNA oligonucleotide comprising the Ebox consensus sequence CAGGTG (SEQ ID NO: 1) and a second DNA oligonucleotide comprising the complementary sequence CACCTG (SEQ ID NO: 2). In some embodiments, the first DNA oligonucleotide comprises the sequence TACGACAGGTGTTGGGA (SEQ ID NO: 3) and the second DNA oligonucleotide comprises the sequence TCCCAACACCTGTCGTA (SEQ ID NO: 4). In some embodiments, the nucleic acid comprises the binding site for a Gli protein transcription factor. In some embodiments, the nucleic acid comprises a first DNA oligonucleotide comprising a Gli protein transcription factor consensus sequence and a second DNA oligonucleotide comprising the complementary sequence. In some embodiments, a first DNA oligonucleotide comprises the sequence CCTGGGTGGTC (SEQ ID NO: 5) and a second DNA oligonucleotide comprises the complementary sequence GACCACCCAGG (SEQ ID NO: 6). In some embodiments, a first DNA oligonucleotide comprises the sequence CTACCTGGGTGGTCTCT (SEQ ID NO: 7) and a second DNA oligonucleotide comprises the complementary sequence AGAGACCACCCAGGTAG (SEQ ID NO: 8).

In some embodiments, the “nucleic acid” in Formula 6 is an oligonucleotide. In some embodiments, the nucleic acid (e.g. DNA or RNA) is single stranded or double stranded. In some embodiments, the nucleic acid is a protein binding site. In some embodiments, the nucleic acid is a transcription factor binding site. In some embodiments, the nucleic acid comprises the binding site for Snail family transcription factors. In some embodiments, the nucleic acid comprises a first DNA oligonucleotide comprising the Ebox consensus sequence CAGGTG (SEQ ID NO. 1) and a second DNA oligonucleotide comprising the complementary sequence CACCTG (SEQ ID NO. 2). In some embodiments, the first DNA oligonucleotide comprises the sequence TACGACAGGTGTTGGGA (SEQ ID NO. 3) and the second DNA oligonucleotide comprises the sequence TCCCAACACCTGTCGTA (SEQ ID NO. 4). In some embodiments, the nucleic acid comprises the binding site for a Gli protein transcription factor. In some embodiments, the nucleic acid comprises a first DNA oligonucleotide comprising a Gli protein transcription factor consensus sequence and a second DNA oligonucleotide comprising the complementary sequence. In some embodiments, a first DNA oligonucleotide comprises the sequence CCTGGGTGGTC (SEQ ID NO. 5) and a second DNA oligonucleotide comprises the complementary sequence GACCACCCAGG (SEQ ID NO. 6). In some embodiments, a first DNA oligonucleotide comprises the sequence CTACCTGGGTGGTCTCT (SEQ ID NO. 7) and a second DNA oligonucleotide comprises the complementary sequence AGAGACCACCCAGGTAG (SEQ ID NO. 8). In some embodiments, the present invention provides a composition comprising a structure of Formula 7:

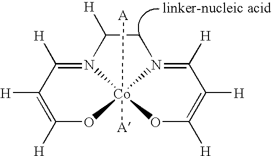

wherein A and A′ are axial ligands.

In some embodiments, the present invention provides a composition comprising a structure of Formula 8:

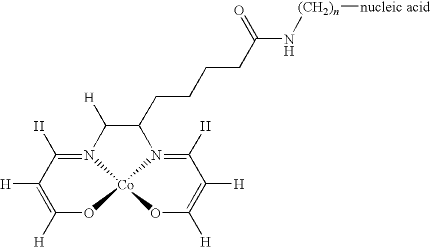

In some embodiments, the present invention provides a composition comprising a structure of Formula 9:

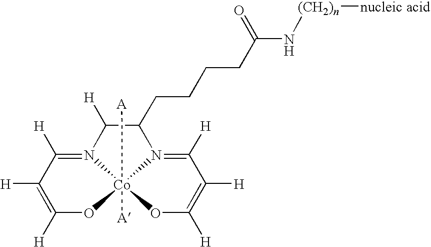

The present invention further provides methods of using the above compositions for research, diagnostic, drug screening, and therapeutic applications. For example, in some embodiments, the methods of inhibiting the activity of a protein are provided, comprising: contacting a protein, or a cell, tissue, or subject having the protein or suspected of having the protein, with a composition described herein. In some embodiments, inhibition comprises inhibiting the DNA binding activity of a DNA binding domain of a protein. In some embodiments, the DNA binding domain is a zinc finger domain. In some embodiments, the protein is a transcription factor. Methods are further provided for targeting a transition metal/Schiff base complex to a protein of interest by conjugating the transition metal/Schiff base complex to a biopolymer, wherein the biopolymer is a binding site for the protein of interest. In some embodiments, methods for inhibiting a transcription factor in a subject or sample are provided, comprising: administering an oligonucleotide-conjugated transition metal/Schiff base complex to a subject or sample, wherein the oligonucleotide targets the transition metal Schiff base complex to the transcription factor. In some embodiments, methods for inhibiting a zinc finger domain-containing protein are provided, comprising: contacting a zinc finger protein and an oligonucleotide-conjugated transition metal Schiff base complex, wherein said oligonucleotide comprises a DNA binding sequence for the zinc finger protein under conditions such that binding of the zinc finger domain to the DNA binding sequence results in exchanging the transition metal of the transition metal Schiff base complex with the zinc of said zinc finger domain. Methods of treating diseases in a subject are further provided, comprising: administering a compound described herein to the subject such that the compound is targeted to a disease promoting transcription factor. Any disease or condition associated with transcription factor activity may be treated. In some embodiments, the disease or condition is cancer.

In some embodiments, the present invention provides a method of inhibiting the activity of a protein comprising contacting the protein with the composition comprising (a) a transition metal coordination element, (b) a linker element, and (c) a biopolymer element, wherein the coordinated transition metal is conjugated to the biopolymer by the linker. In some embodiments, a transition metal is coordinated by the transition metal coordination element. In some embodiments, a cobalt ion is coordinated by the transition metal coordination element. In some embodiments, the present invention provides inhibiting a protein comprising a DNA binding domain. In some embodiments, the protein comprises a zinc finger DNA binding domain. In some embodiments, the protein is a transcription factor. In some embodiments, the DNA binding protein comprises a zinc finger DNA binding domain. In some embodiments, the transcription factors targeted by the compounds are the Snail family of transcription factors. In some embodiments, the transcription factors targeted by the compounds are the Gli family of transcription factors.

In some embodiments, the present invention provides a method of inhibiting a protein comprising contacting said protein with a composition having a structure according to Formula 6. In some embodiments, the protein comprises a DNA binding domain. In some embodiments, the protein is a transcription factor. In some embodiments, the interaction of the cobalt ion with the protein inhibits the activity or disrupts the structure of the protein. In some embodiments, inhibiting the protein inhibits or activates a disease-related pathway.

In some embodiments, the present invention provides a complex comprising a composition having a structure according to Formula 6 and a DNA binding protein. In some embodiments, the DNA binding protein comprises a transcription factor. In some embodiments, the DNA binding protein is inhibited by formation of the complex.

### Definitions

To facilitate an understanding of the present invention, a number of terms and phrases are defined below:

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present invention.

### BRIEF DESCRIPTION OF THE DRAWINGS

The foregoing summary and detailed description is better understood when read in conjunction with the accompanying drawings which are included by way of example and not by way of limitation.

FIG. 1 shows an exemplary synthesis scheme of an exemplary compound. The reagents used at each step are: (a) 1. ICl, NaN₃, MeCN; 2. NaN₃, DMF; (b) 1. H₂, Pd/C, MeOH; 2,4-pentadione, EtOH; (c) CoCl₂, NH₃, O₂, MeOH; (d) 1. N-Hydroxysuccinimide, DCC, DMF; 2. 5′-aminomodified DNA, pH 6.0 MES buffer.

FIG. 2 shows chemical structures of exemplary complexes including Co(III) Schiff base complexes, Co(III)-sb and Co(III)-Ebox conjugate, and the duplex DNA Ebox.

FIG. 3 shows exemplary data demonstrating the identification of Co(III)-Ebox targets. (A) Lysates of blastula stage embryos (lane 1) or embryos with overexpressed Slug protein (lanes 2-6) were incubated with increasing concentrations of Co(III)-Ebox of 0, 0.01, 0.1 and 1 μM for 15 min prior to challenge with a ³²P-labeled Ebox containing Slug DNA probe for 30 min. (B) Lysates of blastula stage embryos injected with Slug, Snail or Sip1 mRNA (lanes 1-9) or Slug, p105, MitF and Lef-1 mRNA (lanes 10-21) were incubated with 0 or 1 μM Co(III)-Ebox for 15 min prior to challenge with ³²P-labeled Slug probe for 30 min and analyzed by EMSA on a native gel. Lanes 1, 4, 7, 10, 13, 16 and 19 are uninjected control embryos incubated with the same labeled oligo as the lysates in the following 2 lanes to demonstrate a lack of non-specific binding. Samples were analyzed by EMSA on a native TBE/acrylamide gel.

FIG. 4 shows an exemplary DNA binding experiment demonstrating DNA-mediated binding selectivity. Lysates of blastula stage embryos injected with Slug mRNA were incubated with increasing concentrations of Co(III)-Schiff base of 0, 1.5, 5, 15, 50, 150 and 500 μM (lanes 2-8) for 15 min, then incubated with ³²P-labeled Slug probe for 30 min and visualized by EMSA. Lysates of embryos injected with Slug (lanes 10 and 11), p105 (lanes 13 and 14), MitF (lanes 16 and 17) or Lef-1 (lanes 19 and 20) mRNA were incubated with 0 or 150 μM Co(III)-Schiff base for 15 min and labeled with ³²P-labeled Slug probe for 30 min. Uninjected embryos are presented in lanes 1, 9, 12, I5 and 18. Samples were separated by EMSA.

FIG. 5 shows an exemplary Western blot highlighting the appearance of epithelial markers in metastatic MDA-MB-231 breast cancer cells. Cells were transfected with 0, 1.0 or 3.0 μM Co(III)-Ebox for 18 h by cationic lipofection. E-cadherin, vimentin and actin Western blots were performed with antibodies for each of the proteins. Actin serves as a loading control.

FIG. 6 shows (A) circular dichroism spectra and (B) melting curve profiles of modified and unmodified DNA.

FIG. 7 shows (A) wildtype and mutant E-cadherin promoter regions and (B) normalized luciferease activity of the E-cadherin construct as a function of Co(III)-Ebox concentration.

FIG. 8 shows western blots for E-cadherin, vimentin, and actin MDA-MB-231 cells treated with increasing concentrations of Co(III)-Ebox.

FIG. 9 shows the chemical structure of an exemplary targeted Co(III) Schiff base complex-DNA conjugate for inhibiting Gli protein transcription factors

FIG. 10 shows gel-shift assays of S2 drosophila cell lysates, transfected with the zinc finger region of Ci, incubated with 10⁻¹² to 10⁻⁷ M of Co(III)-Gli.

FIG. 11 shows Co(III)-Gli injected drosophila embryo with a defect in denticle belt formation at the site of injection.

The figures are discussed in more detail in the context of the Examples section, below.

### DETAILED DESCRIPTION OF EMBODIMENTS

The present invention provides compositions for inhibiting the activity of a target protein. The compositions comprise: (a) a biopolymer element, which targets the composition to the target protein; (b) a transition metal coordination element, which inhibits the target protein's activity; and a linker element, which conjugates the biopolymer and transition metal coordination elements to each other. Compositions of the present invention can be configured to inhibit any protein capable of binding a biopolymer. In preferred embodiments, the biopolymer element comprises a nucleic acid. In these embodiments, the conjugates are configured to target any protein capable of binding to the nucleic acid sequence. The nucleic acid sequence is altered to target specific proteins or classes of proteins. Conjugates of the present invention target DNA binding proteins, transcription factors, enzymes, polymerases, etc. The present invention finds utility in inhibiting proteins which contribute to a wide variety of diseases (e.g. cancer, inflammatory disease, heart disease, neurological disorders, blood disorders, etc.) or metabolic pathways, as well as targeting pathogenic organisms. The present invention finds utility in treating any disease or condition which is regulated by a protein which can be inhibited by a conjugate of the present invention. The present invention can inhibit or activate a pathway by inhibiting a protein which regulates (e g inhibits or activates) that pathway. By conjugating the transition metal coordination element to the consensus binding site for a protein or family of proteins, the transition metal coordination element is delivered within close proximity of the target protein. The transition metal coordination element the inhibits the target protein or disrupts its structure by interacting with key amino acid residues (e.g. histidines (e.g. active site histidines)), exchanging key metal ions with the target protein (e.g. catalytically essential metal ion, metal ions required for proper protein folding, the zinc ion of a zinc finger DNA binding domain, etc.), and/or through other interactions with the target protein, although the present invention is not limited to any particular mechanism of action and an understanding of the mechanism of action is not necessary to practice the present invention.

In some embodiments, the present invention provides transition metal complexes (e.g. Ni, Cd and Co (e.g. Co(III))) in a targeting system to guide the metal center to a reactive protein species (e.g. zinc-finger of a DNA binding protein, active site of an enzyme, etc.). In some embodiments, complexes of a Co(III) Schiff base, a linker, and biopolymer targeting element target, and inhibit or destabilize, a protein of interest. Previous Schiff base complexes are limited to topical application as anti-virals, lack specificity, and target all proteins that contain histidine residues, whereas compounds described herein have more specificity and find use in a variety of applications where regulation of transcription factor activity is desired. In some embodiments, the present invention provides a composition comprising: (a) a transition metal coordination element, (b) a linker element, and (c) a biopolymer element, wherein the coordinated transition metal is conjugated to the biopolymer by the linker.

In some embodiments, a transition metal coordination element is configured to bind one or more transition metals (e.g. Scandium, Titanium, Vanadium, Chromium, Manganese, Iron, Cobalt, Nickel, Copper, Zinc, Yttrium, Zirconium, Niobium, Molybdenum, Technetium, Ruthenium, Rhodium, Palladium, Silver, Cadmium, Lanthanum, Hafnium, Tantalum, Tungsten, Rhenium, Osmium, Iridium, Platinum, Gold, Mercury, Actinium, Rutherfordium, Dubnium, Seaborgium, Bohrium, Hassium, Meitnerium, Darmstadtium, Roentgenium, and Ununbium). In some embodiments, a transition metal coordination element is configured to bind nickel, cadmium, zinc, iron, or cobalt. In some embodiments, a transition metal coordination element is configured to bind cobalt (e.g. Co(II), Co(III), etc.). In some embodiments, the transition metal coordination element is a cobalt metal Schiff base compound (See e.g. U.S. Pat. No. 6,008,190, 5,880,149, and 6,780,856, herein incorporated by reference in their entireties) as a transition metal coordination element.

Cobalt-containing complexes have been shown to have antiviral, antitumor and antimicrobial activities, as well as showing utility in the treatment of inflammation and burns (see U.S. Pat. Nos. 4,866,054, 4,866,053, 5,049,557, 5,106,841, 5,142,076, and 5,210,096, and Wooley et al., Agents and Actions 35:274 (1992), herein incorporated by reference in their entireties).

In some embodiments, the transition metal coordination element coordinates the transition metal using any suitable geometry of the ligand portions of the transition metal coordination element, for example, linear, trigonal planar, tetrahedral or square planar, trigonal bipyramidal or square pyramidal, octahedral or trigonal prismatic, pentagonal bipyramidal, square antiprismatic, or tri-capped trigonal prismatic. In some embodiments, the transition metal coordination element comprises any suitable configuration of carbons, heteroatoms, and hydrogens to coordinate the transition metal.

In some embodiments, the transition metal coordination element comprises one or more Schiff base functional groups. In some embodiments, the transition metal (e.g. cobalt (e.g. Co(II), Co(III)) is coordinated or partially coordinated by one or more Schiff base functional groups.

In some embodiments, the transition metal coordination element adopts the general structure of Formula 2:

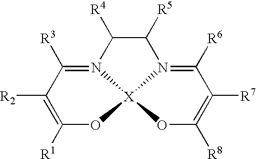

wherein X comprises the transition metal (e.g. cobalt (e.g. Co(II), Co(III))) and R₁-R₈ each may comprise an attachment site for a linker or any other suitable substituent, for example, any of hydrogen, alkyl, hydrophilic organic acid, alkyl amine, amine, alkyl alcohol, alcohol, or aryl.

In some embodiments, the transition metal coordination element adopts the general structure of Formula 4:

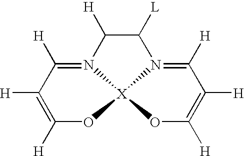

wherein X comprises the transition metal (e.g. cobalt) and L comprises the attachment site for a linker

In some embodiments, the coordinated metal ion (e.g. cobalt) provides a “warhead” functionality. In some embodiments, the warhead is directed to a protein of interest (e.g. a specific location on a protein of interest (e.g. DNA binding domain, catalytic active site, etc.)). In some embodiments, the warhead inactivates or inhibits a protein of interest. In some embodiments, the coordinated metal ion exchanges with a metal ion of a protein of interest (e.g. coordinated cobalt exchanges for the zinc in a zinc finger DNA binding domain), thereby inhibiting the activity of the protein of interest (e.g. DNA binding, enzymatic activity, etc.). In some embodiments, the coordinated metal ion binds to a critical region of a protein of interest thereby inhibiting an activity of the protein of interest (e.g. DNA binding, enzymatic activity, etc.). It should be noted, however, that the present invention is not limited to any particular mechanism of action and an understanding of the mechanism of action is not necessary to practice the present invention.

In some embodiments, a transition metal coordination element (e.g. Formulas 2-5, etc.) coordinates a cobalt ion. The cobalt compounds of the invention utilize either Co(II) or Co(III). Generally, Co(II) compounds have up to four coordination atoms, and may contain a first axial ligand, although it is possible that water molecules may be weakly associated in one or both axial ligand positions. Similarly, Co(III) compounds have up to six coordination atoms of which two are defined as axial ligand positions. Without being bound by theory, in some embodiments, the cobalt compounds of the invention derive their inhibitory activity by the substitution or addition of ligands in the axial positions. The biological activity of the cobalt compounds of the invention results from the binding of a new axial ligand, commonly the nitrogen atom of imidazole of the side chain of histidine. The amino acid serving as a new axial ligand of the cobalt compound is required by the target protein for its biological activity. Thus, proteins such as enzymes that utilize a histidine in the active site, or proteins that use histidine, for example, to bind essential metal ions, are inactivated by the binding of the histidine in an axial ligand position of the cobalt compound, thus preventing the histidine from participating in its normal biological function. When the cobalt is Co(III), the Co(III) complex is synthesized or formulated with two particular axial ligands. Upon interaction with a target protein, the original axial ligand or ligands are replaced by one or more ligands from a protein. This will occur either when the affinity of the protein axial ligand is higher for the cobalt compound as compared to the original axial ligand, or when the new axial ligand is present in elevated concentrations such that the equilibrium of axial ligand binding favors the binding of the new axial ligand from the protein. Thus, Co(III) complexes are made with axial ligands that can be substituted with other ligands. When the cobalt is Co(II), such complexes may, under certain circumstances, have a first axial ligand. The Co(II) compounds of the invention are most commonly synthesized with no axial ligands. Upon interaction with target a protein, certain moieties, such as the nitrogen atom of the imidazole of the side chain of histidine, within the protein can become an axial ligand, resulting in a tightly-bound protein-cobalt compound complex. This occurs when the Co(II) compound, with its four coordinating atoms from the Schiffs base, binds an imidazole moiety, for example, and is oxidized to a Co(III) compound. In one sense, this may be considered a redox reaction, since the Co(II) compound is oxidized to a Co(III) compound upon binding to the protein. Thus, the imidazole axial ligand serves as a fifth coordinating atom, and is tightly bound. Similar interaction with target proteins are achieved with other coordinated transition metal ions. The present invention is not limited to any particular mechanism of action and an understanding of the mechanism of action is not necessary to practice the present invention.

In some embodiments, a transition metal ion coordinated by a transition metal coordination element of the chemical structure of Formula 2 is further coordinated by one or more additional axial ligands. In some embodiments, a transition metal ion coordinated by a transition metal coordination element and two additional axial ligands adopts a chemical structure according to Formula 3:

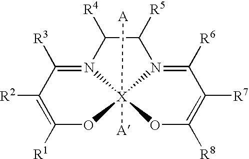

wherein A and A′ correspond to axial ligands that are not covalently attached to the transition metal coordination element. In some embodiments, a metal ion is bound by one additional axial ligand. In some embodiments, a metal ion is bound by two additonal axial ligands. In some embodiments, two axial ligands are part of a single molecule. In some embodiments, a metal ion is bound by axial ligands are two different molecules or functional groups. In some embodiments, axial ligands are selected from 2-methyl imidazole, 4-methyl imidazole, imidazole, ammine, phosphate, analine, pyridine, cysteine, courmarin, etc. In some embodiments, axial ligands comprise phosphorus (P), sulfur (S), nitrogen (N), or oxygen (O) to bind to the central metal ion (e.g. Co(III)). One of skill in the art understands that the axial ligands described herein are not limiting, and any molecules of functional groups capable of coordinating a transition metal ion find use as axial ligands in the present invention.

Without being bound by theory, in some embodiments, compounds of the invention derive their inhibitory activity by exchanging metal ions with the protein of interest. In some embodiments, the metal ion coordinated by the conjugate exchanges with an essential metal ion in the protein of interest, thereby inhibiting the activity or altering the structure of the target protein. In some embodiments, an essential metal ion of a target protein has greater affinity for the transition metal coordination element than for its native binding site in the target protein. The present invention is not limited to any particular mechanism of action and an understanding of the mechanism of action is not necessary to practice the present invention.

In some embodiments, compounds of the present invention comprise a linker region. In some embodiments, the linker connects a coordinated transition metal to a targeting biopolymer. In some embodiments, the linker region covalently connects one coordinated transition metal to one targeting biopolymer. In some embodiments, a linker comprises any molecular element configured to attach one or more transition metal coordination elements (e.g. 1, 2, 3, 4, 5, 6, 7, 8, etc.) to one or more biopolymer elements (e.g. 1, 2, 3, 4, 5, 6, 7, 8, etc.). In some embodiments, the targeting biopolymer is connected to more than one coordinated transition metal by multiple linkers. In some embodiments, more than one targeting biopolymer is connected to more than one coordinated transition metal by multiple linkers. In some embodiments, a linker is configured to attach one transition metal coordination element to one biopolymer element. A wide variety of linkers may be used. In some embodiments, the linker is a single covalent bond. In some embodiments, the linker comprises a linear or branched, cyclic or heterocyclic, saturated or unsaturated, structure having 1-20 nonhydrogen atoms (e.g., C, N, P, O and S); and is composed of any combination of alkyl, ether, thioether, imine, carboxylic, amine, ester, carboxamide, sulfonamide, hydrazide bonds and aromatic or heteroaromatic bonds. In some embodiments, linkers are longer than 20 nonhydrogen atoms (e.g. 21 non-hydrogen atoms, 25 non-hydrogen atoms, 30 non-hydrogen atoms, 40 non-hydrogen atoms, 50 non-hydrogen atoms, 100 non-hydrogen atoms, etc.) In some embodiments, the linker comprises 1-50 non-hydrogen atoms (in addition to hydrogen atoms) selected from the group of C, N, P, O and S (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, or 50 non-hydrogen atoms). In some embodiments, the linker comprises an alkyl chain. In some embodiments, the linker comprises a hexyl chain (i.e., a chain having six repeated units, for example methyl groups). In some embodiments, the linker comprises a polymer (e.g. nucleic acid, polypeptide, lipid, or polysaccharide). In some embodiments, a linker comprises a molecular structure according to Formula 1. In some embodiments, a linker comprises a derivative structure of Formula 1. In some embodiments, a linker comprises a structure according to Formula 1, with one or more additional substituents. In some embodiments, the linker is attached covalently at one end to the coordinating structure and at a second end to a biopolymer. In some embodiments, a linker is enzyme cleavable, such that exposure to specified enzyme cleaves the linker and separates the coordinated transition metal from the targeting biopolymer. In some embodiments, a linker is physically or chemically cleavable, such that exposure to specified conditions (e.g. UV light, heat, pH, etc.) cleaves the linker and separates the coordinated transition metal from the targeting biopolymer. One of ordinary skill in the art will further appreciate that the above linkers are not intended to be limiting. Any linker suitable for connecting the two moieties can be used.

In some embodiments, the biopolymer is a nucleic acid, polypeptide, carbohydrate, lipid, or combination thereof. In some embodiments, a protein of interest of the present invention binds nucleic acids (e.g. DNA binding proteins (e.g. transcription factors), polypeptides (e.g. protein modification enzymes), polysaccharides, and/or lipids (e.g. phospholipid transport proteins, glycolipid transport proteins, retinol binding protein, retinoic acid binding protein, acylCoA binding protein, fatty acid binding proteins, etc.). In some embodiments, the biopolymer is a nucleic acid. In some embodiments, the nucleic acid is composed of DNA, RNA, or combinations thereof. In some embodiments the nucleic acid is double stranded or single stranded. In some embodiments, the nucleic acid is single stranded and hybridized to a second nucleic acid molecule (e.g., through base pairing of complementary bases) or is otherwise associated with a second, complementary nucleic acid molecule (e.g., via crosslinking, intramolecular folding of a self-complementary molecule, etc.). In some embodiments, the nucleic acid comprises the binding sequence for a protein or a class of proteins. In some embodiments, the nucleic acid comprises a binding site which is recognized by the nucleic acid binding domain (e.g. DNA binding domain) of one or more proteins or classes of proteins. In some embodiments, the nucleic acid is recognized by a helix-turn-helix, zinc finger, leucine zipper, winged helix, winged helix-turn-helix, helix-loop-helix, immunoglobulin fold, B3 domain, etc. In some embodiments, the nucleic acid comprises a binding site for one or more transcription factors. In some embodiments, the nucleic acid comprises a consensus sequence for one or more transcription factors. In some embodiments, the nucleic acid comprises a consensus sequence for one or more proteins from a family of transcription factors (e.g. basic-helix-loop-helix, basic-leucine zipper, C-terminal effector domain of the bipartite response regulators, GCC box, helix-turn-helix, homeodomain proteins, lambda repressor-like, srf-like, paired box, winged helix, zinc fingers, multi-domain Cys₂His₂ zinc fingers, Zn₂/Cys₆, Zn₂/Cys₈ nuclear receptor zinc finger, etc.). In some embodiments, the nucleic acid comprises an oligonucleotide which is typically 3-100 nucleotides in length. One of ordinary skill in the art will appreciate that this range expressly embodies compounds of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. One of ordinary skill in the art will further appreciate that the above range is not an absolute limit to the length of an oligonucleotide, but instead represents an exemplary length range. Oligonucleotide lengths falling outside of this range are also included herein provided that the oligonucleotide is amenable the targeting described herein. In some embodiments, the biopolymer of the present invention provides one or more binding sites of a protein of interest (e.g. transcription factor, polymerase, enzyme, etc.). In some embodiments, binding of a protein of interest to a biopolymer of the present invention brings a coordinated transition metal into close proximity of the protein of interest (e.g. a critical region of the protein of interest (e.g. DNA binding domain, catalytic center, etc.)).

In some embodiments, the nucleic acid comprises an Ebox consensus sequence (e.g., CANNTG, CACGTG, CAGGTG (SEQ ID NO:1), etc.). In some embodiments, the nucleic acid comprises complementary strands of an Ebox consensus sequence (e.g., CAGGTG (SEQ ID NO:1) and CACCTG (SEQ ID NO:2), etc.). In some embodiments, the nucleic acid comprises a binding site for one or more Snail family of C₂H₂-type zinc finger transcription factors (e.g. SNAI2, Slug, etc.). In some embodiments, the nucleic acid comprises a binding site consensus sequence for one or more Snail family of C₂H₂-type zinc finger transcription factors (e.g. Snail, Slug, Sip1, etc.). In some embodiments, the nucleic acid comprises a binding site consensus sequence for the Slug transcription factor (TGTTTCCATCCCAACACCTGTCGTATACAA (SEQ ID NO:9)). In some embodiments, the nucleic acid comprises a binding site consensus sequence for the Snail transcription factor (TACGACAGGTGTTGGGA (SEQ ID NO:3)), and TCCCAACACCTGTCGTA (SEQ ID NO:4)). In some embodiments, the nucleic acid comprises a binding site consensus sequence for the Lef-1 transcription factor (TCGAATTCTCACCCTTTGAAGCTCTT (SEQ ID NO:10)). In some embodiments, the nucleic acid comprises a binding site consensus sequence for the NF-κB (GCCTGGGAAAGTCCCCTCAACT (SEQ ID NO:11)). In some embodiments, the nucleic acid comprises a binding site consensus sequence for the MitF (CAGGTG 3′ (SEQ ID NO:12)).

In preferred embodiments, one transition metal coordination element is connected to a biopolymer by one linker. In some embodiments, a transition metal coordination element, linker element, and biopolymer element (e.g. nucleic acid), are attached to form a composition of the present invention according to the chemical structure of Formula 6:

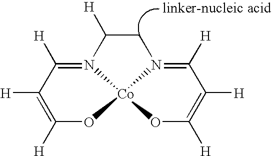

In some embodiments, a transition metal is coordinated by two axial ligands and a transition metal coordination element connected to a biopolymer (e.g. nucleic acid) according to the chemical structure of Formula 7:

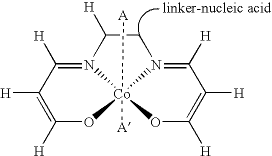

In some embodiments, any suitable axial ligands, linker element, and nucleic acid described herein are used with the structure of Formula 7.

In some embodiments, a transition metal coordination element, linker element, and biopolymer element (e.g. nucleic acid), are attached to form a composition of the present invention according to the chemical structure of Formula 8:

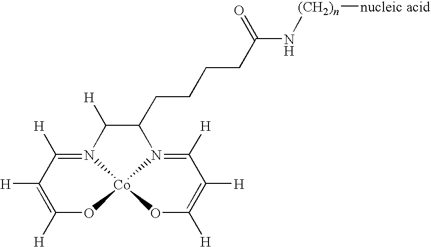

In some embodiments, any suitable nucleic acid described herein is used with the structure of Formula 8.

In some embodiments, a transition metal is coordinated by two axial ligands and a transition metal coordination element connected to a biopolymer (e.g. nucleic acid) according to the chemical structure of Formula 9:

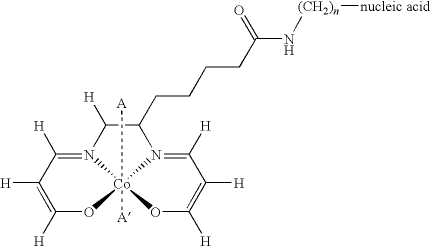

In some embodiments, any suitable axial ligands and nucleic acid described herein are used with the structure of Formula 9.

Any suitable chemistry can be used to attach the linker element to the biopolymer element and transition metal coordination element of the present invention. In some embodiments, a linker comprises functional groups on either end to facilitate attachment to the biopolymer element and transition metal coordination element. In some embodiments, a linker attaches to both the biopolymer element and transition metal coordination element by the same chemistry. In some embodiments, a linker attaches to the biopolymer element and transition metal coordination element using different chemistry. In some embodiments, one of skill in the art will understand suitable chemistries for attachment of the three elements. In some embodiments, maleimide-thiol chemistry provides a means for attaching a linker to a biopolymer element and/or transition metal coordination element. In some embodiments, an oxime linker provides the chemistry for attaching a linker to a biopolymer element and/or transition metal coordination element. In some embodiments, click chemistry provides a means for attaching a linker to a biopolymer element and/or transition metal coordination element (Kolb et al. (2001) “Click Chemistry: Diverse Chemical Function from a Few Good Reactions”. Angewandte Chemie International Edition 40 (11): 2004-2021; Evans (2007). “The Rise of Azide-Alkyne 1,3-Dipolar ‘Click’ Cycloaddition and its Application to Polymer Science and Surface Modification”. Australian Journal of Chemistry 60 (6): 384-395; herein incorporated by reference in their entireties).

In some embodiments, a composition of the present invention is configured to bind a target protein via the biopolymer element, and inhibit or destabilize the target protein via the coordinated transition metal element. In some embodiments, a target protein is configured to bind to the biopolymer element. In some embodiments, target proteins are proteins that bind nucleic acids. In some embodiments, target proteins have nucleic acid binding domains. In some embodiments, target proteins have DNA binding domains. In some embodiments, target proteins have binding specificity for a specific DNA sequence or consensus sequence. In some embodiments, a target protein may be any protein capable of binding a specific DNA sequence (e.g. transcription factors, polymerases, nucleases, histones, etc.). In some embodiments, a target protein is a transcription factor (e.g. Snail family member, Gli family member, etc.).

In some embodiments, the compositions of the present invention are targeted to a protein of interest. In some embodiments, a target protein is a transcription factor, sequence-specific DNA binding factor, or DNA binding protein. In some embodiments, the biopolymer element of a conjugate of the present invention comprises the DNA binding site for a target protein (e.g. transcription factor), thereby targeting the complex to the target protein (e.g. transcription factor). In some embodiments, the target protein binds to the nucleic acid, bringing the coordinated metal ion into proximity of the target protein.

In some embodiments, compositions of the present invention target a DNA-binding domain of a protein. In some embodiments, the DNA-binding domain is selected from the list of helix-turn-helix, zinc finger, leucine zipper, winged helix, winged helix turn helix, or helix-loop-helix. In some embodiments, the DNA-binding domain is a zinc finger. In some embodiments, the DNA-binding domain is a C₂H₂ class zinc finger comprising a pair of cysteine residues and two histidine residues which bind a zinc ion. In some embodiments, the DNA-binding domain is a C₄ or C₆ class zinc finger. In some embodiments, the compounds target a triple-C₂H₂ zinc finger protein, multiple-adjacent-C₂H₂zinc finger protein, and/or separated-paired-C₂H₂ zinc finger protein.

In some embodiments, the target protein comprises a metal ion essential for its activity, folding, or function. In some embodiments, the metal ion of the target protein is exchanged for the metal ion of the transition metal coordination element. In some embodiments, the metal ion of the target protein has a greater affinity for the transition metal coordination element than its binding site in the target protein. In some embodiments, exchange of the target protein metal ion and the metal ion of the present invention inhibits the activity of the target protein. In some embodiments, exchange of the target protein metal ion and the metal ion of the present invention disrupts the structure or folding of the target protein, although the present invention is not limited to any particular mechanism of action and an understanding of the mechanism of action is not necessary to practice the present invention.

In some embodiments, a target protein is an enzyme with a catalytically essential metal ion. In some embodiments, the target protein is a transcriptional enhancer, activator, repressor, etc. In some embodiments, the target protein is a transcription factor which is part of a larger transcription complex.

In some embodiments, the present invention targets an enzyme (e.g. selected from the enzyme classes oxioreductases (e.g. alcohol oxioreductases, aminoacid reductases peroxidases, oxygenases, etc.), transferases (e.g. acyltransferases, glycosyltransferases, transaminases, phosphotransferases, sulfurtransferases, etc.), hydrolases (e.g. peptidases, etc.), lyases, isomerases (racemases, epimerases, cis-trans-isomerases, intramolecular oxidoreductases, intramolecular transferases, intramolecular lyases, etc.), and ligases). In some embodiments, the compounds target a region critical to the function of an enzyme (e.g. active site, substrate binding site, mobile element, etc.). In some embodiments, a biopolymer element directs a conjugate to a specific enzyme or class of enzymes. In some embodiments, an enzyme or class of enzymes interacts with (e.g. binds) the conjugate (e.g. the targeting biopolymer). In some embodiments, a coordinated metal ion is configured to inhibit an enzyme or class of enzymes. In some embodiments, a coordinated metal ion exchanges with a metal ion critical to the function of an enzyme. In some embodiments, a coordinated metal ion becomes coordinated or partially coordinated by an enzyme. In some embodiments, a coordinated metal ion inhibits the activity of an enzyme or class of enzymes. In some embodiments, a coordinated metal ion disrupts a critical aspect of an enzyme (e.g. folding, substrate binding, co-factor binding, activity, etc.).

In some embodiments, compositions of the present invention bind to and inhibit the activity of target enzymes. In some embodiments, the inactivation of the enzyme by the cobalt compound inhibitor is effectively irreversible. In some embodiments, a target enzyme is contacted with a composition of the present invention. The imidazole side chain of an active site histidine binds to the composition as an axial ligand. In the case of Co(II), this occurs with a simultaneous or rapid oxidation of the Co(II) compound to form an enzyme-Co(III) compound complex. This is termed “redox coupling”. In alternative embodiments, the reactive axial ligand from the enzyme is the indole side chain of tryptophan or the side chains of cysteine, methionine. arginine, lysine, asparagine, glutamine, aspartate or glutamate. The availability of these moieties may depend on the pH of the solution containing the protein or enzyme, since in the protonated state these moieties are not good electron donors suitable as axial ligands. Thus, enzymes with these groups within the active site, or enzymes which have functionally important tryptophans, cysteines, or methionines may be inactivated by the compounds of the present invention.

In some embodiments, metalloproteins are inactivated with the compositions of the present invention. Generally, the metals of metalloproteins have ligands such as histidine, cysteine and methionine. If one or more of these residues are inactivated using compositions of the present invention, the binding of the metal atom may be decreased or eliminated, thus reducing or eliminating biological activity. Particular metalloproteins include, but are not limited to, nucleic acid binding proteins such as “zinc finger” proteins and hemerythrin. Zinc finger proteins utilize histidine and cysteine to bind zinc ions (see Berg, Ann. Rev. Biophys. Biophys. Chem 19:405-421 (1990), Berg, Science 232:485 (1986), and Berg, Prog. Inorg. Chem. 37:143 (1989), herein incorporated by reference in their entireties). Zinc finger proteins have been shown to bind nucleic acids and thus play a role in a variety of gene regulatory processes. Zinc finger proteins include transcription factors and other nucleic acid-binding, and gene-regulatory proteins. Zinc finger proteins suitable for inactivation by the compounds of the present invention include the nucleic acid binding domain of steroid and thyroid hormone receptors and the human oncogene product GLI (see Pavletch et al., Science 261:1701 (1993), Kinzler et al., Nature 332:371 (1988)), that contains five zinc finger domains. In a preferred embodiment, one or more of the zinc finger domains utilizes at least one histidine to bind zinc (e.g. two histidines). In some cases the metal is bound exclusively by cysteines. In some embodiments where the metalloprotein is a metalloenzyme, displacement of the active site metal by composition of the present invention modulates enzyme activity. Such metalloenzymes include, but are not limited to, the carboxypeptidases, carbonic anhydrase, thermolysin, collagenase, histidinol dehydrogenase, leukotriene A4 hydrolase, adenosine deaminase, superoxide dismutase, alcohol dehydrogenase, lactate dehydrogenase, stromalycin, aminoacyclase, tryptophanyl-tRNA synthetase, and others known in the art.

In some embodiments, the target protein is a transcription factor that binds to a specific sequence of DNA. In some embodiments, the target protein is a mammalian transcription factor. In some embodiments, the target protein is involved in or associated with a disease (e.g. cancer). In some embodiments, the target protein is involved in or associated with cell motility, cell metastasis, and/or cell migration. In some embodiments, the compounds target a Snail family protein (e.g. Slug, Snail, Sip1, etc.). In some embodiments, the compounds target a Gli family protein.

In some embodiments, protein targets of embodiments of the present invention include Ebox binding proteins (SEQ ID NO:1-2), Snail family members (e.g. Snail (SEQ ID NO:3-4), Slug (SEQ ID NO:9), Sip1, etc.), Gli family members (SEQ ID NO:5-8), Lef-1 (SEQ ID NO:10), NFκB (SEQ ID NO:11), Mit-F (SEQ ID NO:12), etc.

In some embodiments, compositions of the present invention target one or more Ebox binding proteins. In some embodiments, the biopolymer element comprises a Ebox binding sequence such as CANNTG, CACGTG, CAGGTG (SEQ ID NO:1), etc.). In some embodiments, the biopolymer element comprises the complementary strands of an Ebox consensus sequence (e.g., CAGGTG (SEQ ID NO:1) and CACCTG (SEQ ID NO:2). In some embodiment, the binding site sequence facilitates the interaction between the transition metal coordination element and the Ebox binding protein. In some embodiments, interaction of the Ebox binding protein with the biopolymer brings the transition metal coordination element within close proximity of the Ebox binding protein. In some embodiments, metal ion exchange between the transition metal coordination element and the Ebox binding protein alters the activity, structure, and or function of the Ebox binding protein. In some embodiments, binding of the coordinated transition metal to one or more amino acid residues of the Ebox binding protein alters the activity, structure, and or function of the Ebox binding protein. In some embodiments, the complexes of the present invention target Ebox binding zinc finger domains and inhibit DNA binding to the transcription factors. In some embodiments, the complexes are configured to provide a ligand substitution reaction, wherein labile amines are displaced to form the aquo species, and the activated transition metal coordinates the lone pairs on the nitrogenous donors on the imidazole ring of histidines in the zinc finger, displacing the coordinated zinc (Louie & Meade. Proc Natl Acad Sci USA 1998, 95(12), 6663-8, herein incorporated by reference in its entirety); although, the present invention is not limited to any particular mechanism of action and an understanding of the mechanism of action is not necessary to practice the present invention.

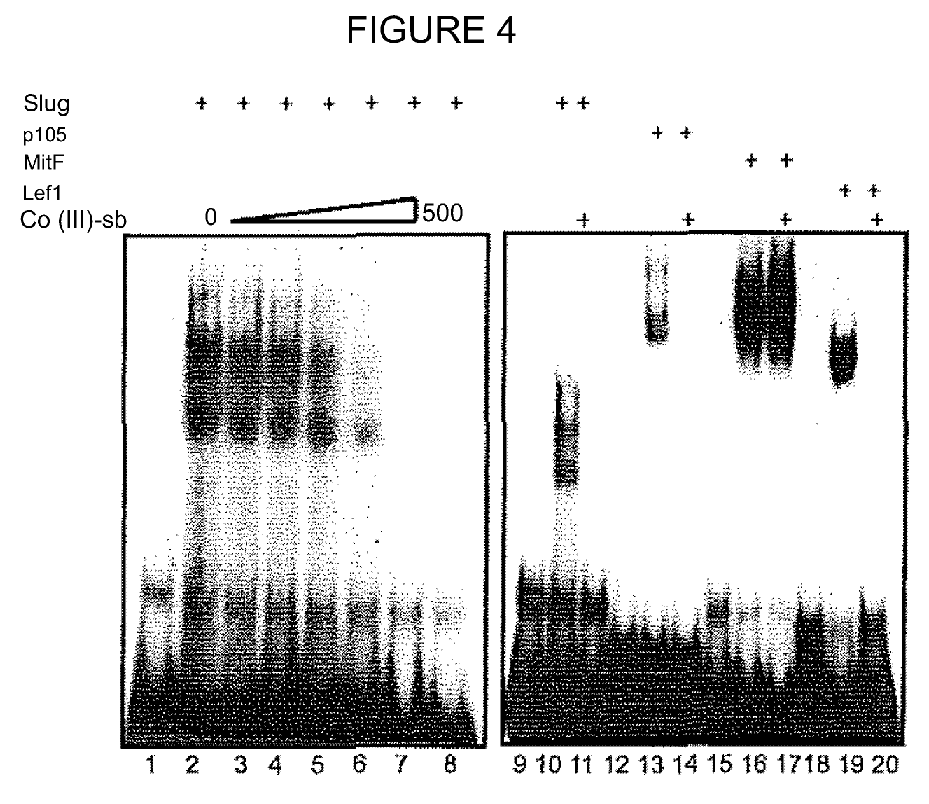

In some embodiments, compositions of the present invention target and inhibit one or more Snail family members. Snail family transcription factors transcription factors are involved in tumor metastasis through the regulation of epithelial-to-mesenchymal transitions (EMTs) (Savagner et al. (1997) J Cell Biol 137(6):1403-1419, Vandewalle et al. (2005) Nucleic Acids Res 33(20):6566-6578, Kuphal et al. (2005) Melanoma Res 15(4):305-313, Blanco et al. (2002) Oncogene 21(20):3241-3246, herein incorporated by reference in their entireties). During EMT, transcriptional repressors down-regulate the expression of proteins involved in cell-cell adhesions characteristic of epithelial cells. Proteins involved in invasion, such as matrix metalloproteinases, are upregulated and cells lose their epithelial characteristics becoming invasive mesenchymal cells (Shiozaki et al. (1996) Cancer 77(8 Suppl):1605-1613,Martinez-Estrada et al. (2006) Biochem J 394(Pt 2):449-457, Peinado (2004) Mol Cell Biol 24(1):306-319, Yang et al. (2005) Cancer Res 65(8):3179-3184, Peinado et al. (2004) Int J Dev Biol 48(5-6):365-375, herein incorporated by reference in their entireties). In some embodiments, the present invention provides compositions and methods that specifically inhibit the activity of one or more Snail family transcription factors (e.g. Snail, Slug, Sip-1, etc.). In some embodiments, transition metal complexes are targeted to Snail family factors. Snail family transcription factors interact with DNA through zinc fingers of the C₂H₂ type with each finger coordinating one zinc ion. Slug, Snail and Sip1 bind to the Ebox consensus sequence CAGGTG (SEQ ID NO:1) in the promoter region of target genes with high specificity to mediate transcriptional repression. Due to their role in EMT, and consequently metastasis, these transcription factors are emerging as targets for new cancer therapeutics (Weinstein & Joe (2006) Nat Clin Pract Oncol 3(8):448-457, herein incorporated by reference in its entirety). A zinc ion coordinated to two histidine and two cysteine residues is integral to the structure and function of zinc finger domains (Hartwig. (2001) Antioxid Redox Signal 3(4):625-634, herein incorporated by reference in its entirety). Transition metals, such as Ni, Cd, Zn, Fe and Co, have a high affinity for biological molecules, as they are rich in electron donors such as phosphates on DNA and the amino acids histidine, lysine and cysteine commonly found in enzyme active sites. The affinity of the coordinated zinc for the histidine and cysteine residues has been used to generate transition metal inhibitors of enzymatic activity as well as DNA-protein interaction (Epstein S P, et al. (2006) B M C Ophthalmol 6:22, Schwartz et al. (2001) J Virol 75(9):4117-4128, Larabee et al. (2005) Chem Res Toxicol 18(12):1943-1954, Watkin et al. (2003) Toxicology 184(2-3):157-178, herein incorporated by reference in their entireties. Displacement of the zinc ion by a Co(III) Schiff base complex has been shown to inhibit DNA binding a zinc finger transcription factor and enzymatic function of non DNA-binding proteins (Louie & Meade (1998) Proc Natl Acad Sci USA 95(12):6663-6668, Bottcher et al. (1997) Inorganic Chem 36:2498-2504, Takeuchi et al. (1999) Bioorg Med Chem 7(5):815-819, Blum et al. (1998) Proc Natl Acad Sci USA 95(12):6659-6662, herein incorporated by reference in their entireties). In some embodiments, transition metal complexes are specific for Snail family factors relative to other proteins (e.g. other zinc containing proteins). In some embodiments, an acetyl acetonate ethylenediimine (acacen) backbone is linked to a modified oligonucleotide containing a consensus sequence (e.g. the Ebox consensus sequence CAGGTC recognized by Snail factors) (Martinez-Estrada et al. Biochem J 2006, 394, (pt 2), 449-57, herein incorporated by reference in its entirety). In some embodiments, modification of DNA oligos by conjugation to transition metal complexes does not alter the secondary structure of the DNA. In some embodiments, the present invention provides oligos configured to be bound by proteins in a concentration dependent manner. In some embodiments, the present invention provides specific oligonucleotides, such that only proteins with a specific binding domain (e.g. zinc finger domain) are configured to bind the specific oligonucleotides (e.g. the Ebox sequence) and are inhibited. For example, experiments performed during the development of embodiments of the present invention demonstrated a functional role for oligos and Co(III) chelate of Co(III)-Ebox of the present invention in the inhibition of DNA-binding of Snail family transcription factors. In some embodiments, the specific inhibitory effects of Co(III)-Ebox for Snail family proteins (e.g. Slug, Snail and Sip1) demonstrates that Co(III)-Ebox find use in therapeutic indications and in the study of epitheial-to-mesenchymal transition (EMT) (e.g. in vertebrate development and epithelial tumor metastasis) (SEE FIG. 4).

In some embodiments, compositions of the present invention target and inhibit one or more Gli family transcription factors (e.g. Gli1, Gli2, Gli3, etc.). The Gli proteins are the effectors of Hedgehog (Hh) signaling and are involved in cell fate determination, proliferation and patterning in many cell types and most organs during embryo development (Ruiz i Altaba A. Development 126 (14): 3205-16, herein incorporated by reference in its entirety). Activation of the Hedgehog pathway leads to an increase in Snail protein expression and a decrease in E-cadherin and Tight Junctions (Xingnan et al. Oncogene. 99 (4): 609-621, herein incorporated by reference in its entirety). Hedgehog signaling also appears to be a crucial regulator of angiogenesis and thus metastasis (Velcheti. Med Hypotheses. 69 (4): 948-949, herein incorporated by reference in its entirety). The Gli transcription factors activate or inhibit transcription by binding to Gli responsive genes and by interacting with the transcription complex. The Gli transcription factors have DNA binding zinc finger domains which bind to consensus sequences on their target genes to initiate or suppress transcription (Sasaki et al. Development 124 (7): 1313-22, herein incorporated by reference in its entirety). In some embodiments, the present invention provides compositions and methods that specifically inhibit the activity of one or more Gli family transcription factors (e.g. Gli1, Gli2, Gli3, etc.). In some embodiments, compositions of the present invention bind to one or more Gli family transcription factors, exchange coordinated metal ions, and thereby inhibit the activity of the transcription factor. In some embodiments, compositions of the present invention affect cell fate determination, proliferation through the inhibition of Gli family transcription factors. In some embodiments, compositions of the present invention inhibit tumor metastasis through the inhibition of Gli family transcription factors. In some embodiments, the targeting sequence 5′-CTACCTGGGTGGTCTCT-3′ (SEQ ID NO:6), the target sequence for the hedgehog signaling pathway protein Gli, is used to target the hedgehog signaling pathway. In some embodiments, the hedgehog targeting sequence is linked to a transition metal complex and the hedgehog sequence targets the transition metal complex to the Gli protein. Similarly, other oligonucleotides may be used to target other proteins to understand, detect, or regulate the associated biological pathways and their corresponding cellular functions.

In some embodiments, compositions of the present invention target and inhibit NF-κB. Aberrant activation of NF-κB is frequently observed in many cancers. Moreover, suppression of NF-κB limits the proliferation of cancer cells. In addition, NF-κB is a key player in the inflammatory response. Hence methods of inhibiting NF-κB signaling has potential therapeutic application in cancer and inflammatory diseases (Garg et al. Leukemia 16 (6): 1053-68.), Sethi et al. Exp. Biol. Med. 233 (1): 21-31, herein incorporated by reference in their entireties). In some embodiments, compositions of the present invention bind to NF-κB, exchange coordinated metal ions, and thereby inhibit the transcriptional regulation activity of NF-κB. In some embodiments, compositions of the present invention limit the proliferation of cancer cells through the inhibition of NF-κB.

In some embodiments, the present invention provides a pharmaceutical composition configured to treat disease (e.g. mammalian diseases, e.g. cancer)). In some embodiments, the present invention targets a redox active transition metal to specific domains (e.g. zinc-finger domain) of specific proteins (e.g. transcription factors (e.g. transcription factors involved in the progression and maintenance of cancer). In some embodiments, the present invention inhibits the activity of proteins involved in regulating pathways related to disease (e.g. cancer, inflammation, heart disease, neuroplogical disorders, blood disorders, etc.). In some embodiments, the present invention provides chemotherapies, for example, preventing metastasis, tumor progression, and/or recurrence. In some embodiments, the present invention provides a vaccine for metastatic cancers. In some embodiments, the present invention is co-administered with other therapeutics and/or pharmaceuticals (e.g. to treat cancer. In some embodiments, compositions of the present invention are administered as a post-cancer therapy (e.g. to inhibit reoccurrence of cancer). In some embodiments, the present invention inhibits the reoccurrence of cancer. In some embodiments, the present invention inhibits the reoccurrence of cancer by arresting migration of cancer cells and/or inhibiting metastasis. In some embodiments, the present invention is administered post-therapy to inhibit the reoccurrence of cancer.

Compositions and methods of the present invention are not limited to pharmacological uses. In some embodiments, the present invention provides clinical, diagnostic, research, and experimental applications. In some embodiments the present invention provides introducing transition metal complexes into model systems including, without limitation, Drosophila melanogaster, Zebrafish, Xenopus laevis, mouse, rat, cell culture, tissue culture, rodents, yeast, and bacteria, to assess the ability of the complexes to regulate transcription factor activity or function and/or to assess the effect of the complexes on cells, tissues, or organisms. In some embodiments, research applications of the present invention provide examination of the molecular mechanism of targeted proteins (e.g. revealing a specific activity, such as transcriptional modulation or inactivation). In some embodiments, research applications of the present invention provide systems for studying protein function (e.g. by inactivating a protein without eliminating the entire protein from the system).

In some embodiments, the present invention provides a research tool to study development, for example in developmental model organisms (e.g. Xenopus laevis, zebrafish, mouse, etc.). In some embodiments, the present invention provides a means for rapid protein level inhibition. In some embodiments, the present invention provides a means for assessing development through inhibition of selected proteins. In some embodiments, the present invention rapidly enters cells and inhibits protein function, thereby providing a mechanism for researching timed cellular event, for example, those that occur during development.

In some embodiments, the present invention provides applications in clinical, research, and diagnostic (e.g, drug screening) applications. In some embodiments, targeted transition metal complexes are used to knock out protein activity (e.g. during animal development) to provide a system for understanding signaling pathways involved in numerous areas of biology (e.g. morphogenesis and differentiation of tissues). In some embodiments, targeted transition metal complexes regulate the function of protein products involved in cancer (e.g. maintenance and progression). In some embodiments, complexes are used to selectively target misregulated protein products (e.g. in tumor tissue).

In some embodiments, the present invention provides compositions and methods for prevention and treatment of cancer, for example those selected from the list of bladder cancer, breast cancer, colon and rectal cancer, endometrial cancer kidney (renal) cancer, leukemia, lung cancer, melanoma, non-Hodgkin lymphoma, pancreatic cancer, prostate cancer, skin cancer, and thyroid cancer. In some embodiments, the present invention provides compositions and methods for prevention and treatment of solid tumor cancers, for example, gastrointestinal cancers, lung cancers, breast cancer, melanomas, etc.

In some embodiments, the present invention provides a pharmaceutical compound comprising oligonucleotide-linked transition metal complexes. In some embodiments, the present invention provides a pharmaceutical compound for the treatment or prevention of cancer. When used for the above purposes, said pharmaceutical compound may be administered via any desired oral, parenateral, topical, intervenous, transmucosal, and/or inhalation routes. The pharmaceutical compound may be administered in the form of a composition which is formulated with a pharmaceutically acceptable carrier and optional excipients, flavors, adjuvants, etc. in accordance with good pharmaceutical practice.

In some embodiments of the present invention, compositions are administered to a patient alone or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert. In another embodiment of the present invention, compositions may be administered alone to individuals suffering from cancer.

Depending on the purpose of administration (e.g. type of cancer being treated), these compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

The composition may be in the form of a solid, semi-solid or liquid dosage form: such as tablet, capsule, pill, powder, suppository, solution, elixir, syrup, suspension, cream, lozenge, paste and spray. As those skilled in the art would recognize, depending on the chosen route of administration, the composition form is determined. In general, it is preferred to use a unit dosage form of the inventive inhibitor in order to achieve an easy and accurate administration of the active pharmaceutical compound. In general, the therapeutically effective pharmaceutical compound is present in such a dosage form at a concentration level ranging from about 0.5% to about 99% by weight of the total composition: i.e., in an amount sufficient to provide the desired unit dose. A therapeutically effective amount of a compound in some method embodiments as described herein used in the present invention may vary depending upon the route of administration and dosage form. Effective amounts of compounds used in some method embodiments of the present invention typically fall in the range of about 0.001 up to 1000 mg/kg/day, and more typically in the range of about 0.1 up to 10 mg/kg/day.

In some embodiments, the pharmaceutical compound may be administered in single or multiple doses. The particular route of administration and the dosage regimen will be determined by one of skill, in keeping with the condition of the individual to be treated and said individual's response to the treatment.

In some embodiments, substituents of a composition of the present invention may be adjusted to provide desirable solubility or other characteristics for administration by any suitable technique.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

For oral administration, tablets containing various excipients such as sodium citrate, calcium carbonate and dicalcium phosphate may be employed along with various disintegrants such as starch and preferably potato or tapioca starch, alginic acid and certain complex silicates, together with binding agents such as polyvinylpyrrolidone, sucrose, gelatin and acacia. Additionally, lubricating agents such as magnesium sterate, sodium lauryl sulfate and talc are often used for tabletting. Solid compositions of a similar type may also be employed as fillers in soft and hard-filled gelatin capsules; preferred materials in this connection include lactose or milk sugar as well as high molecular weight polyethylene glycols. When aqueous suspensions and/or elixirs are desired for oral administration, the active ingredient may be combined with various sweetening or flavoring agents, colorants or dyes, and, if so desired, emulsifying and/or suspending agents as well, together with such diluents as water, ethanol, propylene glycol, glycerin and various like combinations thereof.

The present invention also provides a pharmaceutical composition in a unit dosage form for administration to a subject, comprising a pharmaceutical compound and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants, and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions. Suppositories for rectal administration of the pharmaceutical compound can be prepared by mixing the drug with a suitable nonirritating excipient such as cocoa butter and polyethylene glycols, which are solid at ordinary temperatures but liquid at body temperature and which therefore melt in the rectum and release the drug. Additionally, it is also possible to administer the aforesaid pharmaceutical compounds topically and this may be preferably done by way of cream, salve, jelly, paste, ointment and the like, in accordance with the standard pharmaceutical practice.

### Experimental

### Example 1

### Compositions

Unless otherwise noted, materials and solvents used in development of embodiments of the present invention were purchased from commercial suppliers and used without further purification. Reactions were performed under an atmosphere of N₂ in oven-dried glassware unless otherwise stated. Thin-layer chromatography was performed on Merck 60F 254 silica gel plates. Visualization of developed chromatograms were performed by CAM stain or platinum stain. Flash chromatography was carried out using Fisher Grade 60 A230-400 mesh silica gel. Ion exchange column chromatography was carried out using SP-Sephadex C-25 weak cation exchanger from Pharmacia Biotech. Organic extracts were dried over MgSO₄ and concentrated under reduced pressure. NMR spectra were obtained on a Varian Inova spectrometer at 500 MHz and a Varian Mercury spectrometer at 400 MHz. NMR chemical shifts were reported in ppm and referenced to residual protonated solvent. UV visible spectroscopy was performed on a HP8452A diode array spectrometer thermostated to 37 DC. Mass spectrometry samples were analyzed using electrospray ionization (ESI) using a Varian 1200L Quadrupole MS system, or matrix-assisted laser desorption ionization time of flight (MALDI-TOF) mass spectrometry HPLC analyses were performed on a Varian Prostar 500 (for analysis) system. Mobile phase consisted of 1.5 M ˜Cl with 20 mM Tris buffer—in 0.5% CH₃CN, pH 8 (solvent A) and 20 mM tris buffer in 0.5% CH₃CN, pH 8 (solvent B).

### Example 2

### Synthesis

During development of embodiments of the present invention, the following synthesis strategies were utilized. The following strategies are not limiting, and the present invention should not be limited to any particular synthesis strategy.

### 6,7-Diazido-heptanoie acid (2)

Iodine monochloride (698 mg, 4.3 mmol) was added to a mixture of sodium azide (558 mg, 8.6 mmol) in acetonitrile (10 mL) at 0 DC. The mixture was stirred for 5 min, and 6-heptenoic acid (500 mg, 3.9 mmol) was added slowly. The mixture was stirred at room temperature for 20 h. The reaction mixture was poured into water and extracted with diethyl ether. The organic layer was washed with 5% sodium thiosulfate and water, dried over MgSO₄, and concentrated under reduced pressure. Sodium azide (575 mg, 10.1 mmol) was added to a solution of the crude residue in DMF (5 mL). The mixture was heated at 80 DC for 20 h. The reaction mixture was diluted with water and extracted with diethyl ether. The combined organic layer was washed with water, dried over MgSO₄, and concentrated under reduced pressure. The residue was purified by flash column chromatography over silica gel with ethylacetate:hexane (3:2) as an eluent to give 2 as a yellow oil (622 mg, 87%).

¹H NMR (500 MHz, chloroform-d) δ 11.2 (1H, bs, COOH), 3.2-3.5 (3H, m, CHCH₂N₃), 2.38 (2H, t, J=4.8 Hz, CH₂CO), 1.4-1.6 (6H, m, NHCH₂CH₂CH₂CH₂CH). ¹³C NMR (126 MHz, chloroform-d) 179.97, 77.48, 77.23, 76.97, 61.80, 54.75, 33.74, 31.39, 25.29, 24.21; ESI-MS m/z [M]₊: 210.99.

### 6,7-Bis((E)-((Z)-4-hydroxypent-3-en-2-ylidene)amino)heptanoic acid (3)

A mixture of 6,7-diazido-heptanoie acid (2) (622 mg, 8.78 mmol) in methanol (50 mL) was hydrogenated on palladium carbon using a hydrogenator at 45 psi for 12 h. The mixture was filtered and concentrated under reduced pressure. The crude mixture was dissolved in hot ethanol (15 mL) and added slowly to a solution of 2,4-pentadione in ethanol (2 mL) over 2 h using a dropping funnel. The mixture was stirred overnight and concentrated under reduced pressure. The crude residue was purified by recrystallization in hot acetonitrile to give 6,7-Bis((E)-((Z)-4-hydroxypent-3-en-2-ylidene)amino)heptanoic acid (3) as a pale yellow solid. (1.79 g, 63%)

¹H NMR (500 MHz, dimethylsulfoxide-d₆) δ 10.75 (1H, bs, OH), 10.65 (1H, d, J=5.25 Hz, OH), 4.90 (1H, s, CH═C(OH)CH₃), 4.89 (1H, s, CH═C(OH)CH₃), 3.63 (1H, m, NCHCH₂), 3.37 (2H, m, CH₂N), 2.17 (2H, bs, CH₂NHCO), 1.84 (6H, s, 2×C(OH)CH₃), 1.82 (6H, s, CHN═CCH₃), 1.2-1.5 (6H, m, COCH₂CH₂CH₂CH₂CH); ¹³C NMR (126 MHz, chloroform-d) δ 194.04, 175.19, 163.70, 163.51, 95.74, 54.03, 48.22, 34.38, 33.27, 29.30, 29.49, 25.10, 19.31, 18.97.

[Co(III)(heptanoic acido)(acacen)(NH₃)2]Cl (Co(III)-Schiff Base)

Cobalt(II) chloride was added under nitrogen atmosphere to a solution of 6,7-Bis((E)-((Z)-4-hydroxypent-3-en-2-ylidene)amino)heptanoic acid (3) (495 mg, 1.17 mmol) in degassed methanol (5 mL). The reaction mixture was heated at 50 DC and stirred for 5 h. A saturated ammonia solution was prepared by bubbling ammonia gas through methanol, and this saturated solution (5 mL) was added to the reaction mixture. After stirring for 2 h, the reaction mixture was opened to air, and approximately 200 mg of charcoal was added to the mixture. The reaction mixture was stirred with oxygen bubbling overnight. The mixture was filtered to remove charcoal, and concentrated under reduced pressure. The crude residue was purified by ion-exchange column chromatography over Sephadex C-25 cation exchange resin. The resin was swollen in deionized water overnight and poured into a glass column. The product was eluted with deionized water to collect the brown product band and 1% ammonium hydroxide solution was added. The concentration of ammonium hydroxide solution was gradually increased from 1% to 10% to collect each band. The collected fractions were analyzed by ESI-MS and ¹H NMR. The first band eluted with deionized water was confirmed to be Co(III)-Schiff base and was obtained as a brown solid by freeze drying (335 mg, 69%).

¹H NMR (500 MHz, methanol-d₄) (j 5.11 (1H, s, CH═C(OH)CH₃), 5.05 (1H, s, CH═C(OH)CH₃), 3.74 (1H, m, NCHCH₂), 3.68 (2H, d, J=7 Hz, CH₂N═C), 2.28 (6H, s 2×C(OH)CH₃), 2.23 (2H, m, CH₂CO), 2.13 (3H, s, CHN═CCH₃), 2.12 (3H, s, CH₂N═CCH₃), 1.4-1.7 (6H, m, NHCH₂CH₂CH₂CH₂CH); ¹³C NMR (126 MHz, dimethyl sulfoxide-d₆) Ii 177.74, 177.05, 169.29, 168.14, 97.31, 96.35, 63.41, 56.30, 49.19, 34.18, 25.82, 25.77, 22.87, 22.81; ESI-MS m/z [M−2NH₃]: 381.1, [M+Na−2NH₃]⁺: 403.1.

### Co(III)-oligonucleotide Conjugate(Co(III)-Ebox) (Compound 2)

5′-aminomodified DNA (2.33 J. 1 mol) and the unmodified complementary strand (2.0 μmol) were purchased from IDT DNA (Coralville, Iowa). Each of the complementary DNA strands was suspended in 100 mM MES buffer (pH 6.0, 1 mL). A solution of Co(III)-Schiff base (2.49 mg, 6.0 μmol) and N-hydroxysuccinimide (4.14 mg, 36 μmol) in DMF (200 μL) was treated with DCC (7.43 mg, 36 μmol). The reaction mixture was stirred at room temperature for 1 h. The reaction was monitored by ESI-MS to confirm the formation of activated ester of Co(III)-Schiff base. The reaction mixture was added to 260 μL of the solution of 5′aminomodified DNA (600 nmol) in solution and stirred at room temperature for 24 h. To this solution, 600 nmol of the complementary strand was added and the mixture was heated to 95° C. for 5 min and cooled slowly. The mixture was purified by Microspin G50 to remove excess amount of Co(III)-Schiff base and other coupling reagents and freeze-dried. The crude residue was further purified by anion-exchange HPLC using the following conditions: semi-preparative Dionex DNAPac PA100 column (9×250 mm), 10-60% buffer A (1.5 M ˜Cl, 20 mM Tris, 0.5% CH₃CN, pH=8) into buffer B (20 mM Tris, 0.5% CH₃CN, pH=8) solution over 40 min. Co(III)-Ebox (1) was identified by its absorbance at 260 urn with visible bands due to the MLCT states of the cobalt complex at 336 nm. The retention time of Co(III)-Ebox was approximately 27 min. Following HPLC purification and lyophilization, the fractions containing Co(III)-Ebox were desalted using a Sep-pak cartridge (Waters Corporation, MA) with 60% MeOH/water after the washing with water. The concentration of the conjugate was determined by calculating the Co(III) concentration by inductively couple plasma mass spectrometry (ICP-MS) using the X Series ICP-MS (Thermo Scientific, MA) with 25% yield.

### Example 3

### Methods

The following methods were used in development of embodiments of the present invention. The following methods should not be viewed as limiting.

### MALDI-TOF Mass Spectrometry Analysis

Molecular weights of Co(III)-Ebox were determined by matrix-assisted laser desorption/ionization-time of flight mass spectrometry (MALDI-TOF MS) on a Perseptive Biosystems Voyager Pro DE spectrometer using a 2,4,6-trihydroxyacetophenone (THAP)/ammonium citrate matrix, calibrated by standard polythymine oligonucleotides.

Co(III)-Ebox calculated: [M]/4: 2783.7, found: 2738.9

### Inhibition of Zinc-Finger DNA-Binding

Co(III)-Ebox interacts with three of the transcription factors involved in epithelial mesenchymal transitions, Slug, Snail and Sip1 (SEE FIG. 4). To verify specificity of this interaction transcription factors that could be inhibited by the Co(III) chelate alone, the oligo alone or by non-specific interactions, were also examined. p105 contains a zinc finger region that could interact with untargeted Co(III). MitF can bind the Ebox consensus sequence through a basic helix-loop-helix region, could be inhibited by excess oligo (Goding et al. Genes Dev 2000,14(14),1712-28, herein incorporated by reference in its entirety). Lef-1 contains neither a zinc-finger region, nor specificity for Eboxes and is used to verify that the effects are not due to surface interactions or other non-specific binding. Although Co(III)-Ebox inhibits Slug, Snail and Sip1 at 1 μM incubation concentration, p105, MitF and Lef-I were not inhibited (SEE FIG. 4). These results reveal effective targeting of Co(III)-Ebox to Snail family members with no off-target effects. Effective inhibition requires both a zinc-finger region in the protein for interaction with the Co(III) metal, and binding to the Ebox sequence which will bind to the oligonucleotide.

### Co(III)-Schiff Base is Not Selective in Target Inhibition

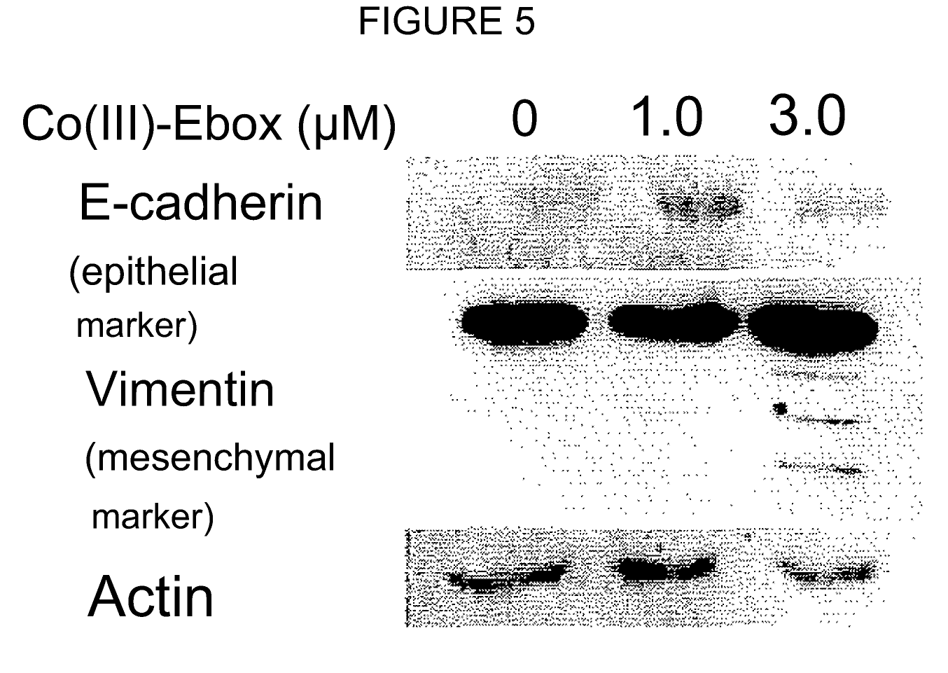

In the absence of a targeting moiety, such as an oligonucleotide, multiple cobalt complexes can interact with a single protein, although only one is required for complete inhibition of function (Louie & Meade. Proc Natl Acad Sci USA 1998,95(12), 6663-8, Blum et al. Proc Natl Acad Sci USA 1998, 95(12), 6659-62, Takeuchi et al. Bioorg Med Chem 1999, 7(5), 815-9, herein incorporated by reference in their entireties). The minimal concentration at which Co(III)-Schiff base inhibits Slug DNA-binding was determined. The Co(III)-Schiff base complex requires 150-fold excess to achieve comparable inhibition of Slug which is achieved by Co(III)-Ebox at 1 μM (Figure H). When DNA binding by p105 and Lef-1 is inhibited, MitF is not (SEE FIG. 5). This is compared to 1 μM, that is sufficient for inhibition by Co(III)-Ebox, which does not inhibit transcription factors that are not targeted by the Ebox moiety (SEE FIG. 4). It is not surprising that at high concentrations Co(III)-Schiff base can inhibit p105 and Lef-1, as both factors contain amino acid residues in the DNA-binding region with electron-donating atoms that can bind with Co(III)-Schiff base to alter the protein structure. In p105, there are both cysteine and histidine residues in the zinc finger regions that have an affinity for Co(III) (Toledano et al. Mol Cell Biot 1993, 13(2), 852-60, herein incorporated by reference in its entirety). Similarly, Lef-1 contains a methionine residue at position 13 important for intercalation into DNA that can interact with Co(III)-Schiff base (Balaeff et al. Proteins 1998, 30(2), 113-35, herein incorporated by reference in its entirety).

### Co(III)-Ebox Affects Neural Crest Induction and Migration

Co(III)-Ebox (1 μM Co(III)-Ebox) was coinjected with β-galactosidase RNA into one side of a stage 1 Xenopus laevis embryo. The uninjected side serves and an internal control for normal embryonic development. The neural crest markers Slug, Sox10 and Twist were used for in situ hybridization to visualize the neural crest. Embryos were cultured in 0.1×MMR at 14 C. In situ hybridization was performed as described (Bellmeyer et al. Dev. Cell 4 (2003), pp. 827-839, herein incorporated by reference in its entirety). Beta-galactosidase activity was visualized with Red-gal.

The side of the embryo injected with Co(III)-Ebox showed deficiency in neural crest induction at stage 18 as seen by deficiency of Sox10 staining for the neural crest (SEE FIG. 4). After stage 20 the neural crest cell population undergoes an epitheial-to-mesenchymal transition (EMT) and migrates to form other tissues including the cranial cartilage and the peripheral nervous system. Post-EMT, the neural crest population shows defect in migration as seen by the lack of defined structures that form on the uninjected side (SEE FIG. 4). The results are consistent with the removal of Slug using antisense RNA (Carl et al. Dev Bio 1999, 213(1), 101-15, herein incorporated by reference in its entirety).

### Changes in EMT Markers in Metastatic Breast Cancer Cells

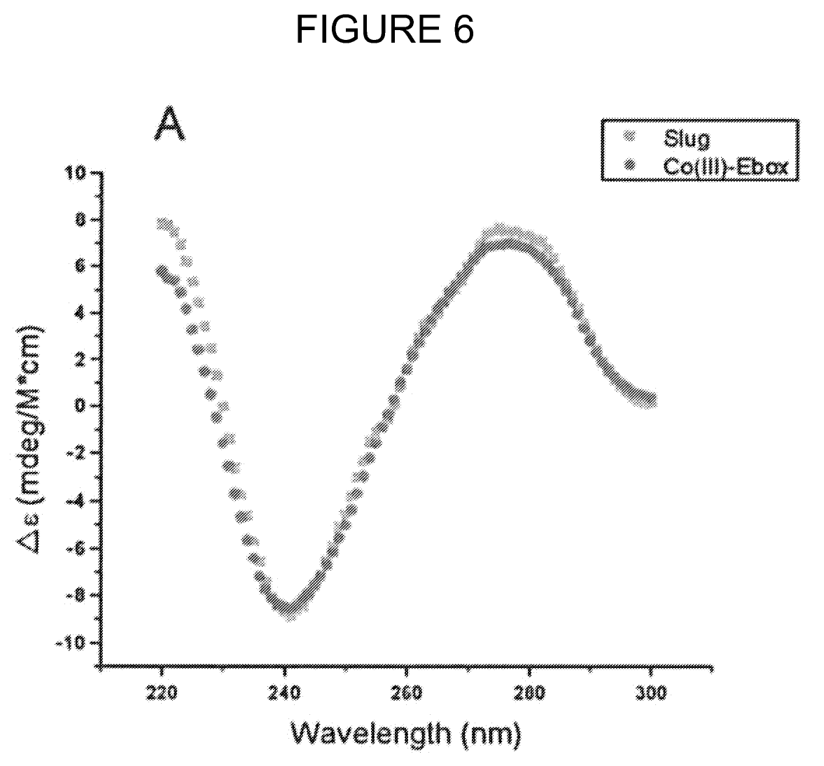

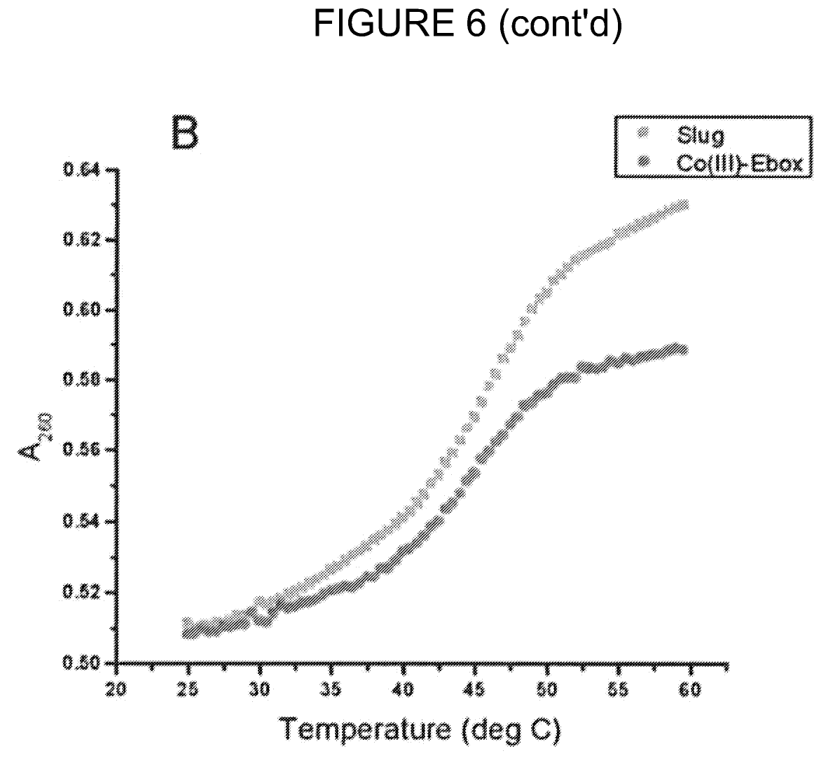

Metastatic breast cancers often express Slug, Snail or Sip1 and have high levels of the mesenchymal marker vimentin and none of the epithelial protein E-cadherin, which is a component of tight junctions (SEE FIG. 6). The metastatic breast cancer cell line MDA-MB-231 expresses Snail and is known to be invasive. MDA-MB-231 cells were treated with 1.0 and 3.0 μM Co(III)-Ebox for 18 h using cationic lipofection. Protein expression levels for E-cadherin and vimentin were determined using Western blotting (SEE FIG. 6). With the addition of Co(III)-Ebox, E-cadherin expression was elevated and vimentin begins to degrade after 18 h. This suggests that with repeated treatment with Co(III)-Ebox the mesenchymal phenotype of these are reversed into an epithelial phenotype by inhibiting the regulator of EMT, Snail.

### Circular Dichroism Spectroscopy

Circular dichroism (CD) measurements were performed on a Jasco Model J-715 spectrometer with 150 W air-cooled Xenon lamp as light source. CD spectra were collected at 25° C. in a 1 cm path length cell at band width 1 nm, data pitch 0.2 nm and response time of 2 s. The concentrations of Co(III)-Ebox and Slug control were 1 μM in 300 mM NaCl/20 mM PBS buffer solutions (pH 7.0).

### Thermal Denaturation

Tₘ measurements were acquired at 260 nm on an Agilent 8453 UV-visible spectrophotometer with a 1 cm optical path length. The temperature was increased by increments of 0.5° C. by a Peltier temperature controller, with a hold time of 1 minute. Measurements were acquired in a solution of 0.1×SSC (150 mM NaCl, 15 mM NaCitrate), with a duplex concentration of 2 μM. The Tₘ for each of the duplexes was determined as the first integral of the curve.

### Embryo Preparation and Constructs

Pigmented and albino eggs were obtained and fertilized using standard protocol (Bellmeyer et al. (2003) Dev Cell 4(6):827-839, herein incorporated by reference in its entirety). All embryos are staged using the Niewenkoop-Faber method. Embryos were injected into one-cell at stage 1 in 0.4× modified ringer's (MR) solution with 3% Ficoll then transferred to 0.1×MR until harvesting. mRNA was transcribed in vitro using the SP6 Message Machine Kit (Ambion, Austin, Tex.). Concentrations of mRNA injected into onecell at the 2-cell stage range from 5-50 pg. XSip1 in the vector pCS2+ was obtained from A. Eisaki (Eisaki (2000) Biochem Biophys Res Commun 271(1):151-157, herein incorporated by reference in its entirety). cDNA was generated using PCR amplification and a high fidelity polymerase (Pfu; Roche, Indianapolis Ind.). cDNAs were cloned into a pCS2 variant that adds 5 myc tags to either the N- or C-terminus. All constructs were confirmed by sequencing using a ABI 3730 high-throughput DNA Sequencer.

### Electrophoretic Mobility Shift Assays

1.25 pmol of annealed oligonucleotide (IDT USA; Coralville, Iowa) was labeled at the 5′ end with ³²P-ATP (Amersham; Picstaway, N.J.) by T4 polynucleotide kinase according to the manufacturer's protocol (Gibco BRL Life Technologies; Paisley, UK). Free nucleotides were removed using ProbeQuant G-50 micro columns (GE Healthcare, UK). Duplex oligonucleotide sequences used were as follows:

Slug:

| | |
|---|---|
| 5′ TGTTTCCATCCCAACACCTGTCGTATACAA 3′ | (SEQ ID NO: 9) |
| Lef-1 : | |
| 5′ TCGAATTCTCACCCTTTGAAGCTCTT 3′ | (SEQ ID NO: 10) |
| NF-KB (Promega): | |
| 5′ GCCTGGGAAAGTCCCCTCAACT 3′ | (SEQ ID NO: 11) |
| MitF: | |
| 5′ CAGGTG 3′ | (SEQ ID NO: 12) |

(Vallin J, et al. (2001) J Biol Chem 276(32):30350-30358, herein incorporated by reference in its entirety)

For in vitro assays, whole embryo lysates were prepared by lysing 5 stage 8 embryos in 100 μL of lysis buffer (1×PBS, 1% NP-40 and Complete protease inhibitor cocktail tablets; Roche, Indianapolis, Ind.). In the EMSA 5 μL of embryo lysate was combined with 25 μg/mL poly dI/dC (Sigma; St. Louis, Mo.),5 μL of EMSA binding buffer (20 mM Hepes pH 7.6, 150 mM KCl, 3 mM MgCl₂, 0.5 mg/mL BSA, 10% glycerol and 0.2 mM ZnSO₄), and indicated amounts of Co(III)-Ebox, which were incubated at room temperature for 15 minutes. After the incubation, 2 μL of radiolabeled oligonucleotide probe was added and incubated at room temperature for an additional 30 min. Protein-DNA complexes were resolved on a 5% TBE/acrylamide gel. In vivo assay embryos were collected after 6 h of incubation at 18° C. and lysed as above. The EMSA binding reaction used 5 μL of lysate was combined with 25 μg/mL poly dI/dC, 5 μL of EMSA binding buffer and incubated at room temperature for 5 minutes. Radiolableled oligonucleotide probe was added and incubated at room temperature for 30 minutes. Protein-DNA complexes were resolved on a 5% TBE/acrylamide gel.

### Example 4

### Design and Synthesis of a Transcription Factor Targeted Co(III) Schiff Base Complex

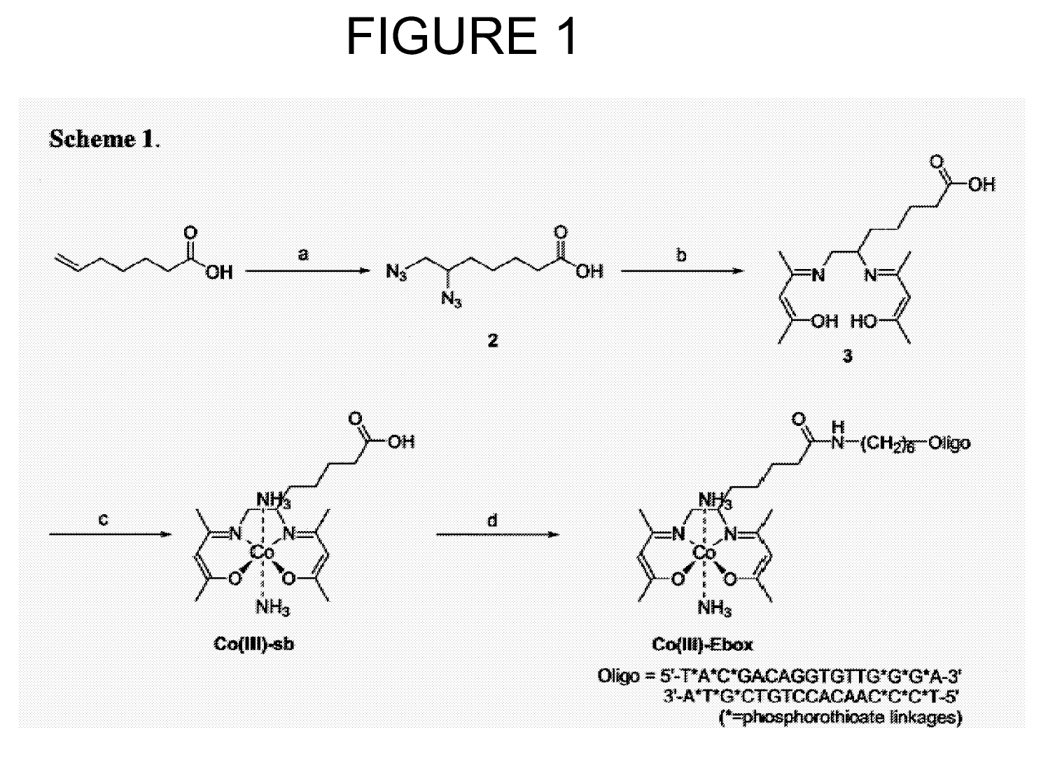

In experiments performed during development of embodiments of the present invention, Co(III) Schiff base complex conjugated to a DNA oligonucleotide that targets Ebox-binding zinc finger transcription factors was synthesized and characterized (SEE FIG. 1). The Co(III) complex was prepared by the reaction of 6-heptenoic acid with sodium aizde and iodine monochloride to give 6-azido-7-iodo-heptanoic acid (2) in 92% yield. The diazide (3) was formed upon reaction of 2 with excess sodium azide in 23% yield. Compound 2 was reduced by hydrogenation and the crude diamine was condensed with 2,4-pentadione to obtain the Schiff base ligand, 4, in 22% yield. Compound 4 was metallated using Co(II) chloride in methanol under a nitrogen atmosphere followed by in situ addition of a methanolic ammonia solution. Bubbling oxygen overnight through the mixture in the presence of charcoal oxidizes the cobalt complex from Co(II) to Co(III) in 69% yield.

Characterization of Co(III)-Schiff base indicates the presence of at least two isomers. Due to resonance within the ligand substructure, this compound can exist in the imine/enol conformation, the enamine/ketone conformation, or a combination thereof. A stereogenic center at the base of the asymmetric arm compounds the complexity of the molecule, leading to at least eight possible conformations. The high-resolution mass spectrum shows a parent peak [C₁₇H₂₅CoN₂O₄] at 380.12160 (calc. 380.1152), while the de-metalled complex is found at 323.20336 (calc. 323.1976). However, the MLCT region of the UV-vis spectrum of Co(III)-Schiff base shows a band at 325 nm accompanied by shoulder at approximately 355 nm, suggesting at least two electronically different metal environments.

The ¹H NMR spectrum contained multiple signals for the olefinic protons (5.27-5.01 ppm). The DQ-COSY shows multiple couplings of the protons adjacent to the Schiff base nitrogens (3.3-4.2 ppm) indicating more than a single isomer. The ¹³C NMR spectrum shows five major carbonyl peaks and two minor peaks; only see 3 peaks would be observed if a single isomer were present. Co(III)-Schiff base exists in several isomeric forms, indistinguishable by these spectroscopic techniques. The mixture of isomers was used to generate Co(III)-Ebox derivatives and its conformation will not affect the axial geometry of the cobalt center.

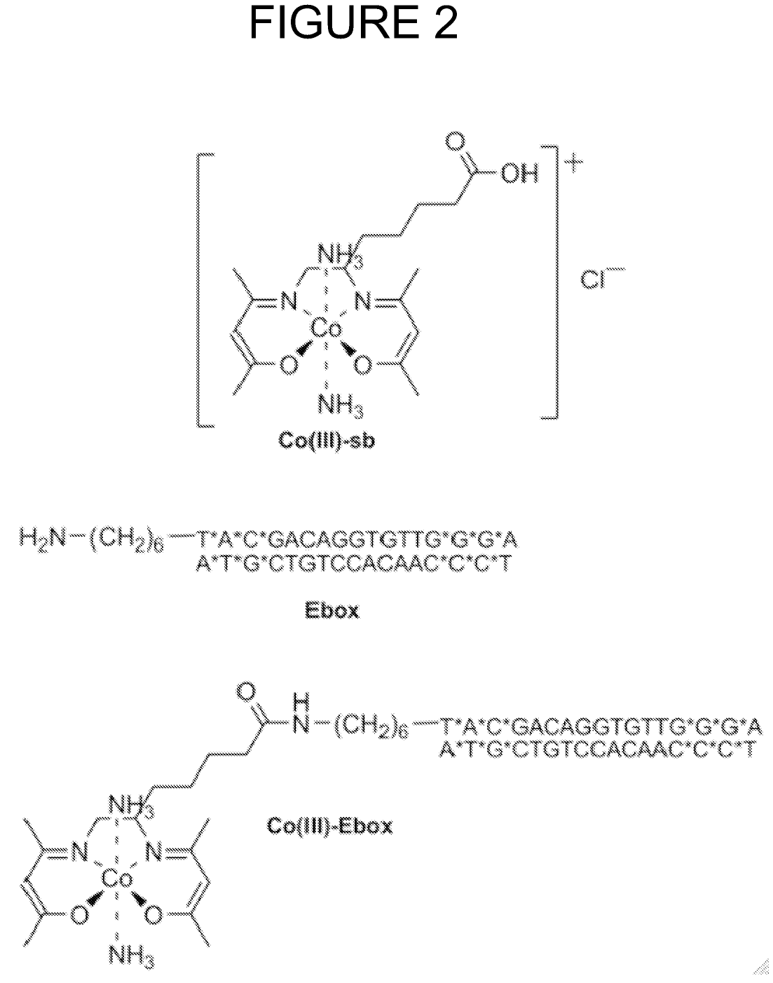

The Co(III) Schiff base complex, Co(III)-Schiff base, was coupled to the oligonucleotide, Ebox (5′-T*A*C*GACAGGTGTTG*G*G*A-3′), containing a six carbon amino-terminated linker at the 5′ end of one of the strands, containing three phosporothioate linkages at both the 3′ and 5′ ends of both strands. This modification prevents degradation by nucleases (as indicated by *, Ebox in bold). Co(III)-Schiff base was coupled to the oligonucleotide using N,N′-dicyclohexyl carbodiimide (DCC) and N-hydroxysuccinate (NHS). The complementary strand of DNA was annealed by heating to 95° C. for five minutes and cooling slowly overnight. The resulting complex, Co(III)-Ebox, was purified using a Sephadex G-25 spin column, and high performance liquid chromatography (HPLC) to yield 7.25 nmol (14.5%) of pure product (SEE FIG. 2).

### Example 5

### Duplex DNA Secondary Structure Evaluation

Native B-DNA conformation of the DNA is essential for a high affinity interaction between the oligonucleotide and the target protein. The secondary structure and strength of the duplex of Ebox and Co(III)-Ebox were examined by circular dichroism (CD) and melting profiles to ensure that modification with a metal chelate in Co(III)-Ebox has not altered the structure (SEE FIG. 6). The negative peak at 250 nm, and the positive peak at 280 nm are characteristic of B-DNA, demonstrating that the secondary structure of the oligonucleotide attached to Co(III)-Ebox is not altered as a result of the conjugation to a Co(III) chelate (SEE FIG. 6A).

To further characterize the duplex DNA, melting points were determined using UV spectroscopy. Co(III)-Ebox DNA and unmodified Ebox DNA were heated in 0.1×SSC from 25.0° C. to 60.0° C. (SEE FIG. 6B). The melting point for Ebox and Co(III)-Ebox were determined to be 46.0° C. and 44.7° C., respectively. The similarity in the shape of the curve and the proximity of the melting points demonstrates that the DNA attached to Co(III)-Ebox maintains its native integrity, allowing the Co(III)-Ebox complex to selectively disrupt the function of its biological target.

### Example 6

### Selectivity of Co(III)-Ebox for Slug, Snail and Sip1

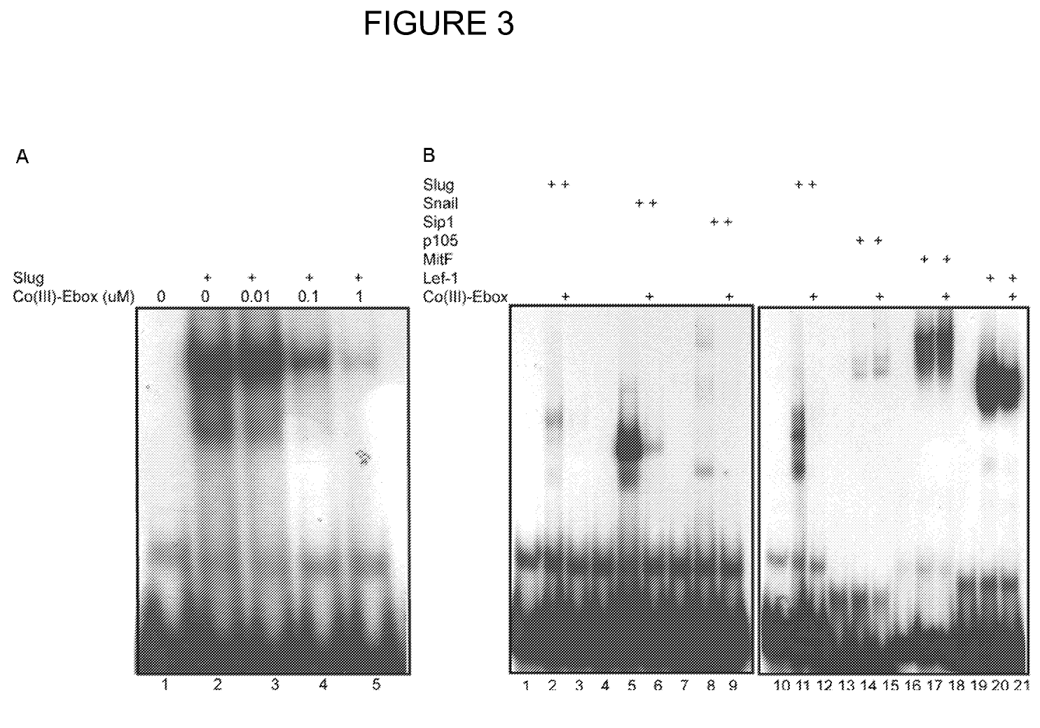

The oligonucleotide of Co(III)-Ebox contains an Ebox sequence, CAGGTG, designed to target the Snail family of transcription factors (SEE FIG. 3). Investigation of the ability of Co(III)-Ebox to bind to the zinc finger region of Slug was conducted by electrophoretic mobility shift assay (EMSA) such that the concentration of Co(III)-Ebox added to each sample was increased. Partial inhibition of DNA-binding was achieved at 100 nM and complete inhibition of DNA-binding at 1 μM. The function of zinc fingers is inhibited by Co(III) Schiff base complexes via a dissociative ligand exchange mechanism. Co(III) has high affinity towards the nitrogen in the imidazole ring of a histidine residue in the zinc finger (Louie & Meade (1998) Proc Natl Acad Sci USA 95(12):6663-6668, herein incorporated by reference in its entirety). This is an irreversible reaction and inhibition of DNA-binding is concentration dependent. These results confirm that Co(III)-Ebox interacts with Slug, deactivating the zinc finger region and inhibiting the proteins' ability to bind DNA.

Slug, Snail and Sip1 have been implicated in having a role in EMT during metastatic progression of epithelial tumors. That Co(III)-Ebox interacts with three of the transcription factors involved in EMT is advantageous as it eliminates the response to more than one molecular mediator of the process. To verify specificity of this interaction transcription factors that could be inhibited by the Co(III)-Schiff base, the oligo alone or by non-specific interactions, were also examined. p105 contains a zinc finger region that could interact with untargeted Co(III)-Schiff base. MitF can bind the Ebox consensus sequence through a basic helix-loop-helix region, could be inhibited by excess oligo. Lef-1 contains neither a zinc finger region, nor specificity for Eboxes and is used to verify that the effects are not due to surface interactions or other non-specific binding.

Although Co(III)-Ebox inhibits Slug, Snail and Sip1 at 1 μM incubation concentration, p105, MitF and Lef-1 were not inhibited. These results reveal effective targeting of Co(III)-Ebox to Snail family members with no off-target effects. Effective inhibition requires both a zinc finger region in the protein for interaction with the Co(III) metal, and binding to the Ebox sequence which will bind to the oligonucleotide.

### Example 7

### Comparison of Ebox, Co(III)-Schiff Base and Co(III)-Ebox Efficacy In Vivo

Confirmation of the requirement for both the oligo and Co(III) chelate moieties (Co(III)-Ebox) for specificity of zinc finger inactivation was achieved by comparing the efficacy of Co(III)-Ebox, Ebox duplex DNA and Co(III)-Schiff base with Slug RNA at varying concentrations and examining DNA-binding after embryos have developed to the blastula stage. Only Co(III)-Ebox inhibits Slug function at an injected concentration of 1 μM. Both Co(III)-Schiff base and Ebox DNA have been shown to bind Slug, however the affinity of these interactions are not on the same order of magnitude. Ebox DNA binding is reversible and has a dynamic on/off rate, while Co(III)-Schiff base requires several orders of magnitude higher concentration to have an inhibitory effect. Even at 10 fold excess (10 μM) Slug is not inhibited by either Ebox or Co(III)-Schiff base. This demonstrates that the effects of Co(III)-Ebox are not mediated by either the oligo or the Co(III) chelate alone but require both for targeted inhibition. Since Co(III)-Ebox maintains inhibitory effects of Slug when injected in vivo, this indicates that Co(III)-Ebox can mediate downstream effects on gene expression resulting in changes in development in the neural crest specific to Slug, Snail and Sip1.

### Example 8

### Co(III)-Ebox Inhibits the Activity of Snail

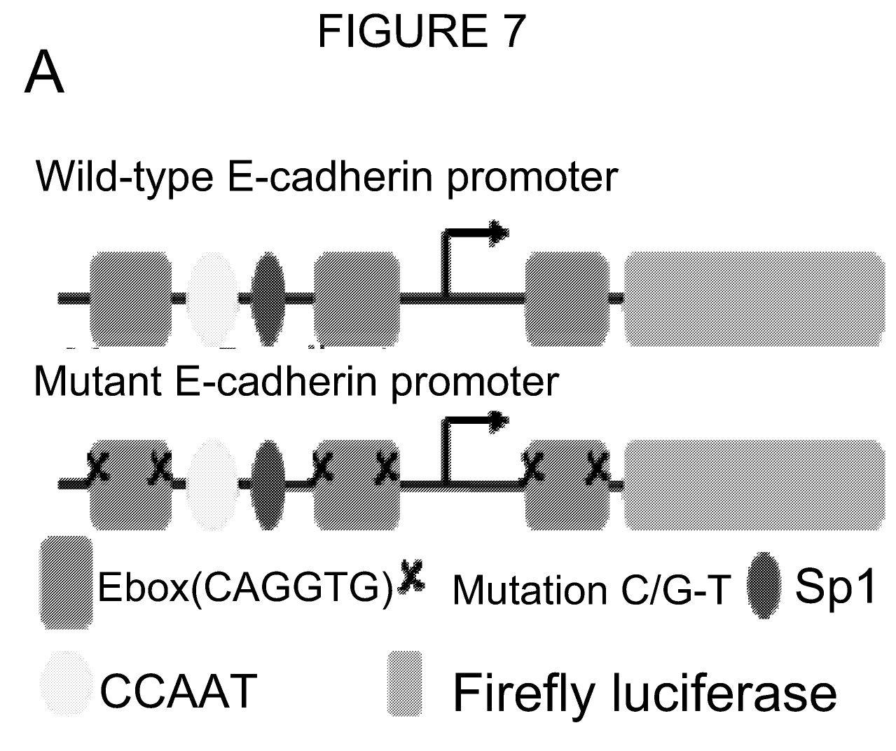

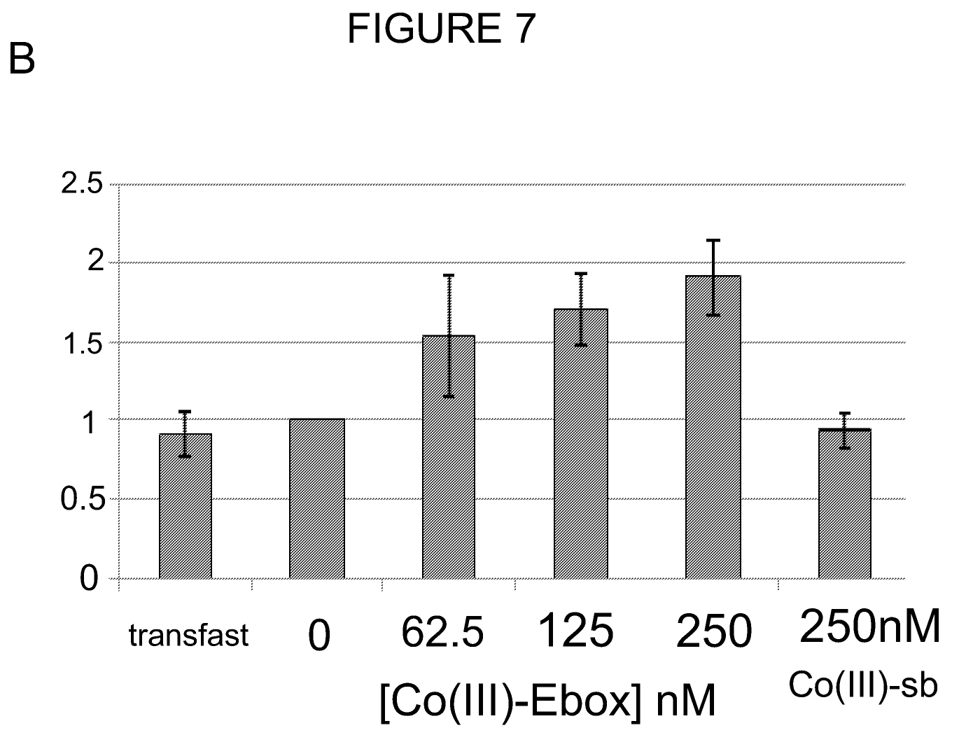

Experiments performed during development of embodiments of the present invention demonstrate that Co(III)-Ebox effectively inhibits the transcriptional repression activity of Snail (SEE FIG. 7). MDA-MB-231 metastatic breast cancer cells express Snail1 in the nucleus. A firefly luciferase plasmid with the E-cadherin promoter region was added upstream of the transcriptional start site Snail (SEE FIG. 7A). Binding of

Snail to the E-boxes thereby repress luciferase transcription and therefore luciferase activity. Up to 250 nM of Snail is removed from the E-boxes in the E-cadherin promoter region by added Co(III)-Ebox, as demonstrated by an increase in light output (SEE FIG. 7B). Co(III)-sb did not affect Snail transcriptional activity.

### Example 9

### Co(III)-Ebox Modulates Snail Transcription to Reverse Markers of EMT

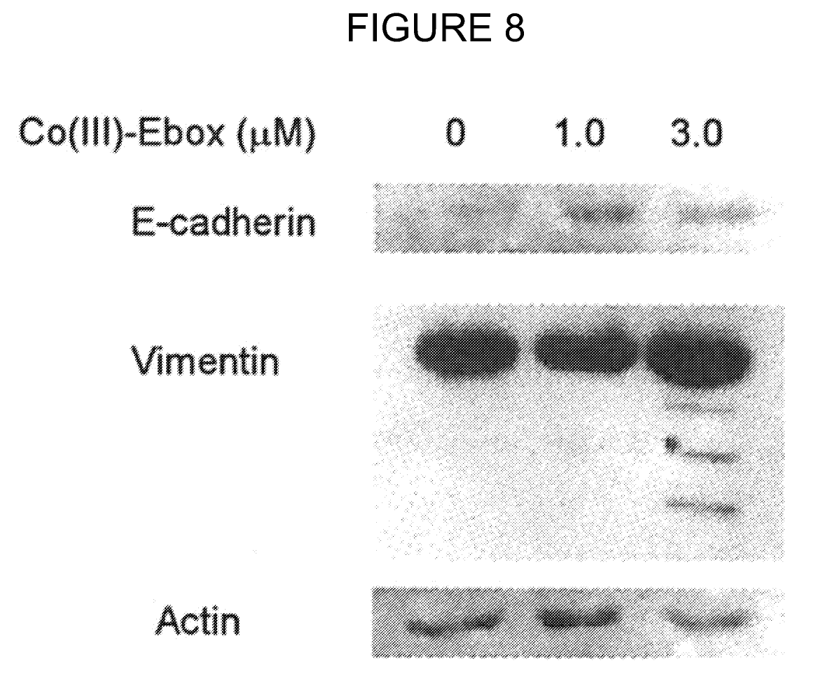

Experiments performed during development of embodiments of the present invention demonstrate that Co(III)-Ebox modulates Snail transcription to reverse markers of EMT. MDA-MB-231 cells treated with increasing concentrations of Co(III)-Ebox show a change in gene expression. A western blot shows an increase in E-cadherin protein in whole cell lysates with additional Co(III)-Ebox up to 3 uM (SEE FIG. 8). E-cadherin is an epithelial marker that is repressed by Snail during EMT. Therefore, this marker is low in metastatic cells. Vimentin is a mesenchymal marker and has high expression in metastatic MDA-MB-231 cells (0 uM Co(III)-Ebox). With the addition of Co(III)-Ebox, vimentin shows characteristic degradation products. Therefore, genetic changes are seen in metastatic breast cancer cells indicative of a loss of the mesenchymal (migratory and invasive) phenotype and a reversion to an epithelial phenotype with adition of Co(III)-Ebox.

### Example 10

### Inhibition of Gli/Ci Zinc Finger Transcription Factors

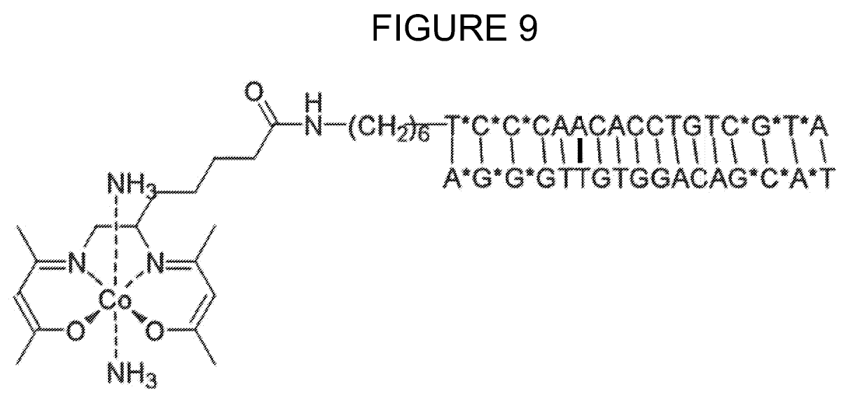

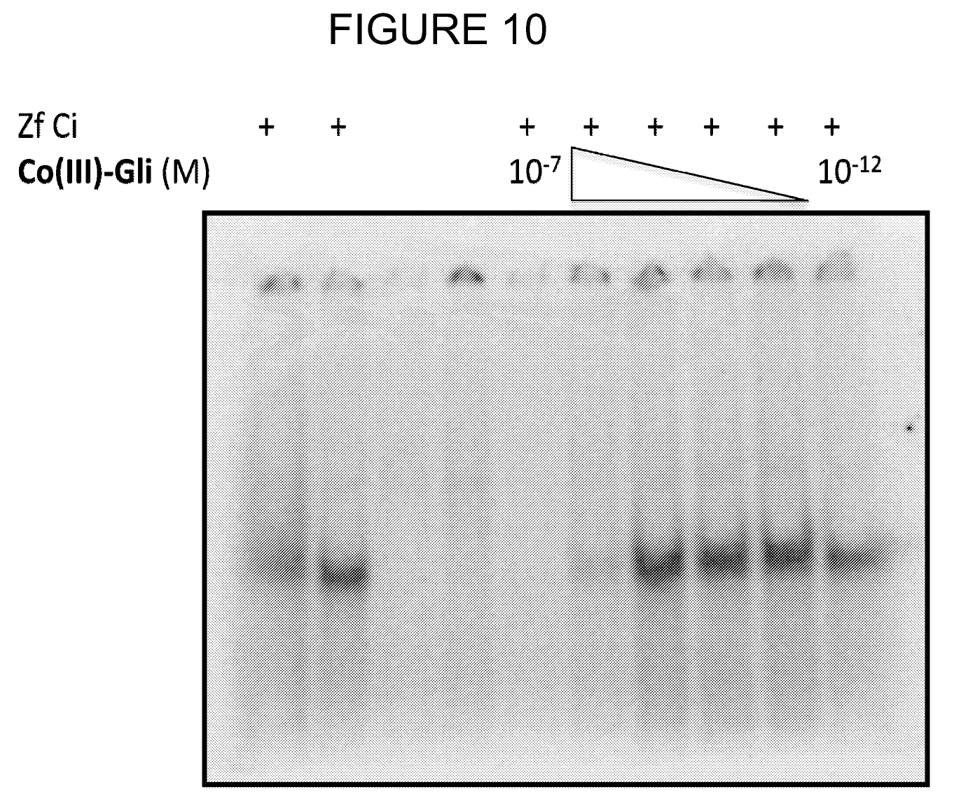

The Hedgehog-Gli signaling pathway is central to normal prostate development. Aberrant activation of the pathway correlates with metastasis and poor prognosis. The Gli protein transcription factors are the effectors of the Hedgehog-Gli pathway. Experiments were performed during development of embodiments of the present invention to test Co(III) Schiff base complex-DNA conjugates (SEE FIG. 9) as inhibitors of Gli protein function (e.g. as a cancer therapeutic). S2 drosophila cells were transfected with the zinc finger region of Ci, and the lysates were incubated with 10⁻¹² to 10⁻⁷ M Co(III)-Gli complex (SEE FIG. 9). The results were visualized on gel-shift assays, and demonstrate clear inhibition of DNA binding (SEE FIG. 10).

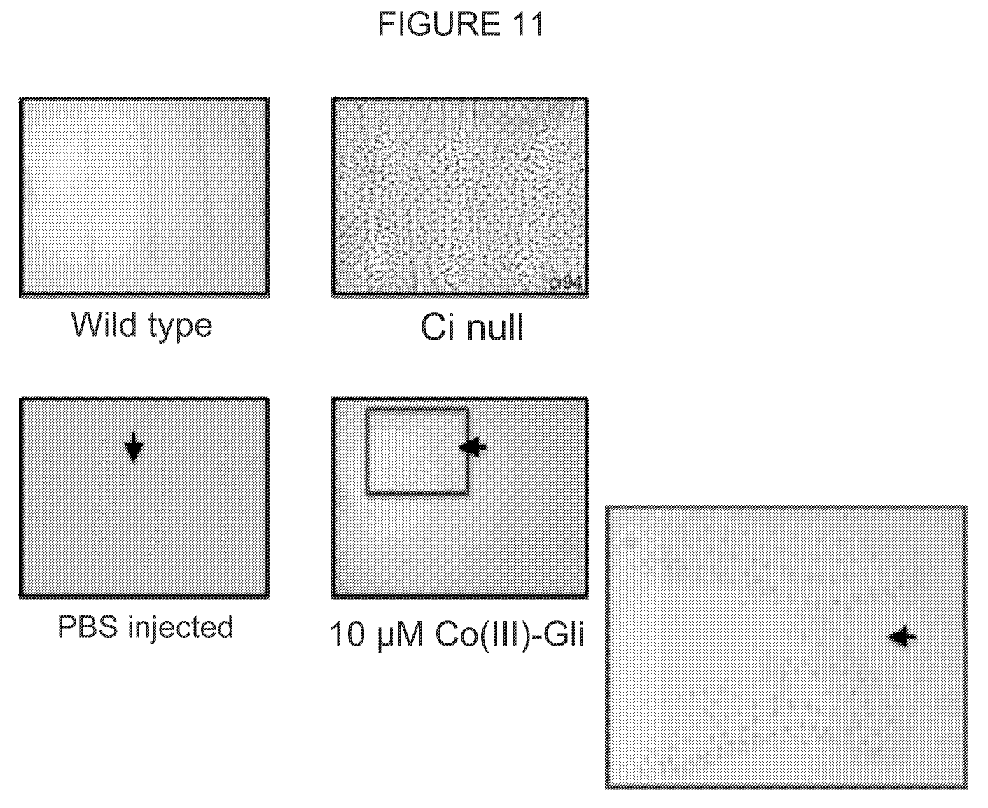

Experiments were performed during development of embodiments of the present invention demonstrate that Co(III)-Gli inhibits Hedgehog mediated denticle belt patterning in Drosophila larvae. Co(III)-Gli injected embryo shows defect in denticle belt formation at the site of injection (SEE FIG. 11).

Experiments were performed during development of embodiments of the present invention demonstrate specificity of Co(III)-Gli for inhibition of Hedgehog signaling. Additionally, experiments demonstrate that the specificity seen in Co(III)-Ebox can be extended to other signal transduction pathways.

We claim:

1. A composition comprising:
a) a biopolymer element;
b) a linker element, wherein said linker comprises a structure comprising Formula 1: 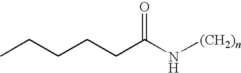
wherein n is an integer between 1 and 10; and
c) a transition metal coordination element, wherein said transition metal coordination element comprises a structure comprising Formula 2: 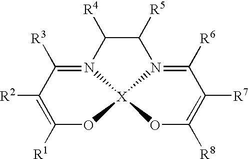
wherein:
X comprises a transition metal;
R₁ comprises hydrogen, alkyl, hydrophilic organic acid, alkyl amine, amine, alkyl alcohol, alcohol, aryl, or an attachment site for said linker;
R₂ comprises hydrogen, alkyl, hydrophilic organic acid, alkyl amine, amine, alkyl alcohol, alcohol, aryl, or an attachment site for said linker;
R₃ comprises hydrogen, alkyl, hydrophilic organic acid, alkyl amine, amine, alkyl alcohol, alcohol, aryl, or an attachment site for said linker;
R₄ comprises hydrogen, alkyl, hydrophilic organic acid, alkyl amine, amine, alkyl alcohol, alcohol, aryl, or an attachment site for said linker;
R₅ comprises hydrogen, alkyl, hydrophilic organic acid, alkyl amine, amine, alkyl alcohol, alcohol, aryl, or an attachment site for said linker;
R₆ comprises hydrogen, alkyl, hydrophilic organic acid, alkyl amine, amine, alkyl alcohol, alcohol, aryl, or an attachment site for said linker;
R₇ comprises hydrogen, alkyl, hydrophilic organic acid, alkyl amine, amine, alkyl alcohol, alcohol, aryl, or an attachment site for said linker; and
R₈ comprises hydrogen, alkyl, hydrophilic organic acid, alkyl amine, amine, alkyl alcohol, alcohol, aryl, or an attachment site for said linker;
and wherein at least one of R₁, R₂, R₃, R₄, R₅, R₆, R₇, and R₈, comprises an attachment site for said linker;
wherein said coordinated transition metal element is conjugated to said biopolymer by said linker.

2. The composition of claim 1, wherein said biopolymer comprises nucleic acid.

3. The composition of claim 2, wherein said nucleic acid comprises the binding site for a DNA binding protein.

4. The composition of claim 1, wherein R₅ comprises an attachment site for said linker, and each of R₁, R₂, R₃, R₄, R₆, R₇, and R₈ comprise hydrogen.

5. The composition of claim 1, wherein the coordinated transition metal is selected from nickel, cadmium, zinc, iron and cobalt.

6. The composition of claim 5, wherein said coordinated transition metal comprises cobalt.

7. The composition of claim 1, further comprising one or more axial ligands complexed with the transition metal X.

8. A composition comprising the structure of Formula 6:

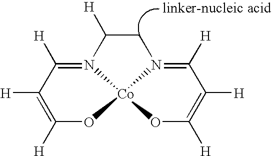

9. The composition of claim 8, wherein said linker comprises the structure of Formula 1.

10. The composition of claim 8, wherein said nucleic acid comprises a binding site for a DNA binding protein.

11. The composition of claim 9, wherein said nucleic acid comprises a binding site for a transcription factor.

12. The composition of claim 10, wherein said nucleic acid comprises a binding site for Snail family transcription factors.

13. The composition of claim 11, wherein said nucleic acid comprises a first DNA oligonucleotide comprising the Ebox consensus sequence CAGGTG and a second DNA oligonucleotide comprising the complementary sequence CACCTG.

14. The composition of claim 8, further comprising one or more axial ligands complexed with said cobalt.

15. A method of inhibiting a protein comprising contacting said protein with a composition of claim 8.

16. The method of claim 15, wherein said protein comprises a DNA binding domain.

17. The method of claim 16, wherein said protein is a transcription factor.

18. The method of claim 15, wherein interaction of the cobalt ion of said composition with said protein inhibits the activity or disrupts the structure of said protein.

19. The method of claim 15, wherein inhibiting said protein inhibits or activates a disease-related pathway.

20. A complex comprising the composition of claim 8 and a DNA binding protein.


SEQUENCE LISTING (12 sequences)

SEQ ID NO: 1 - DNA, 6 bp, Artificial Sequence
  Synthetic
caggtg

SEQ ID NO: 2 - DNA, 6 bp, Artificial Sequence
  Synthetic
cacctg

SEQ ID NO: 3 - DNA, 17 bp, Artificial Sequence
  Synthetic
tacgacaggtgttggga

SEQ ID NO: 4 - DNA, 17 bp, Artificial Sequence
  Synthetic
tcccaacacctgtcgta

SEQ ID NO: 5 - DNA, 11 bp, Artificial Sequence
  Synthetic
cctgggtggtc

SEQ ID NO: 6 - DNA, 11 bp, Artificial Sequence
  Synthetic
gaccacccagg

SEQ ID NO: 7 - DNA, 17 bp, Artificial Sequence
  Synthetic
ctacctgggtggtctct

SEQ ID NO: 8 - DNA, 17 bp, Artificial Sequence
  Synthetic
agagaccacccaggtag

SEQ ID NO: 9 - DNA, 30 bp, Artificial Sequence
  Synthetic
tgtttccatcccaacacctgtcgtatacaa

SEQ ID NO: 10 - DNA, 26 bp, Artificial Sequence
  Synthetic
tcgaattctcaccctttgaagctctt

SEQ ID NO: 11 - DNA, 22 bp, Artificial Sequence
  Synthetic
gcctgggaaagtcccctcaact

SEQ ID NO: 12 - DNA, 6 bp, Artificial Sequence
  Synthetic
caggtg